(19) United States
(12) Patent Application Publication
(10) Pub. No.: US 2009/0276042 A1
(43) Pub. Date: Nov. 5, 2009

(54) Biological Polysiloxanes

(75) Inventors: Timothy Charles Hughes, Lysterfield (AU); John Stuart Wilkie, Viewbank (AU); Justine Leigh Jeffery, Mitcham (AU); Xuan Thi Thanh Nguyen, Springvale (AU); Xiaojuan Hao, Glen Waverley (AU)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: Vision CRC Limited, Kensington (AU)

(21) Appl. No.: 12/296,998

(22) PCT Filed: May 3, 2007
(86) PCT No.: PCT/AU2007/000582

§ 371 Date: Oct. 13, 2008

Related U.S. Application Data
(60) Provisional application No. 60/796,936, filed on May 3, 2006.

(51) Int. Cl. A61F 2/16; C07F 7/08; C07F 7/10; B29D 11/00

(52) U.S. Cl. 623/6.56; 556/439; 556/418; 556/420; 264/1.1

(57) ABSTRACT

The present invention relates to a macromonomer having a polydimethylsiloxane backbone that has a mol % dimethyl siloxanes, b mol % siloxanes substituted with -K-RIM, c mol % siloxanes substituted with -K-RIM-Z and d mol % siloxanes substituted with -L-Z, and in which the terminal siloxane groups are tri-substituted with R, wherein RIM is a refractive index modifying group; Z is a free radically polymerisable group; K is a spacer group; L is optional and is a spacer group; each R is independently selected from an RIM, a lower alkyl group, hydrogen or Z; and a is a molar percentage of the macromonomer which is in the range of from 0 to 95 mol %; b is a molar percentage of the macromonomer which is in the range of from 5 to 99 mol %; c is a molar percentage of the macromonomer which is in the range of from 0 to 2 mol %; and d is a molar percentage of the macromonomer which is in the range of from 0 to 2 mol %; with the proviso that c and d are not both 0 mol %.

### FIELD OF THE INVENTION

The present invention relates to siloxane macromonomers and polymers formed therefrom suitable for use as biomedical devices. In particular, the siloxane macromonomers are suitable precursors for forming injectable, in situ curable, accommodating intraocular lenses.

### BACKGROUND OF THE INVENTION

Currently known intraocular lenses (IOLs) include non-deformable, foldable and expansible lenses, which may be formed from materials such as acrylics, hydrogels or polysiloxanes. These IOLs are implanted by making an incision in the cornea and inserting a preformed IOL. To minimise trauma during implantation, foldable and expansible IOLs have been developed. These lenses may be rolled up and inserted through a small tube, which allows a smaller incision to be made in the cornea. For example, dehydrated hydrogels can be used with small incision techniques. Hydrogel lenses are dehydrated before insertion and naturally rehydrated once inside the capsular sac. To be suitable as IOLs, these deformable lenses require not just appropriate optical properties, but also mechanical properties, such as structural integrity and elasticity, to permit them to deform during implantation and then regain their shape in vivo. However, such IOLs are not capable of accommodating when in vivo, due to their rigidity, and so are not an optimal solution for correction of presbyopia.

To further develop IOLs and reduce surgical incisions to below 1.5 mm, techniques utilising injectable IOLs have been suggested. Injectable IOLs would be implanted by lens filling or refilling procedures, such as Phaco-Ersatz. In such a procedure the natural material of the lens is extracted while the lens capsule-zonule-ciliary body framework is maintained. The intact lens capsule is then refilled by injecting a low viscosity material into the empty capsular bag. The material may then be cured in situ. In this process the capsular bag is used to form the shape of the lens. Provided the elasticity of the refilling material is sufficiently low, the lens shape can then be manipulated by the ciliary muscles and zonules as occurs with the natural lens. Consequently, such injectable IOLs are able to accommodate in vivo.

Apart from problems with in situ curing, such as controlling the crosslinking process and finding clinically acceptable conditions, there has been a struggle to develop polyorganosiloxane compositions for use as injectable IOLs. Injectable IOL materials need to have a suitable viscosity for injection, a suitable refractive index, suitable mechanical characteristics after curing, i.e. modulus, good transparency, be biocompatible, including having minimal extractables, and be sterilisable.

The properties, such as viscosity, modulus and extractables, for an injectable, in situ curable, accommodating intraocular lens differ from those required for deformable IOLs. Consequently, materials useful in deformable IOLs are by no means suitable for use as injectable IOLs.

For example, polydimethylsiloxane (PDMS) has been employed as a material in foldable or deformable IOLs. In the injectable IOL context though, PDMS has been found to have a relatively low viscosity and thereby a tendency to leak out of the injection site (i.e. the capsular bag) before curing. To address this deficiency, high viscosity polysiloxanes have been added to the PDMS reaction mix. However, a drawback of high viscosity silicones is that they can entrap air bubbles, which can impair the optical quality of the resulting product. Also, they are difficult for the surgeon physically to inject in a very delicate environment, often requiring substantial force. In addition, it has been found that polyorganosiloxanes having a high fraction of dimethylsiloxane units may have an unacceptable low specific gravity with the undesired result that the injected lens material will float on any aqueous layer present in the capsular bag. In such a case, it will be difficult to fill the capsular sac completely and will require the surgeon to manually express intra-capsular water in order to maintain the correct lens shape during the filling and curing process.

Alternative polysiloxanes, produced by polymerisation of aromatic-based siloxane macromonomers, for use as deformable IOLs are disclosed in WO 03/040154. WO 03/040154 teaches that the polysiloxanes described in that specification have a relatively high RI of 1.45 or greater and are biocompatible. However, such polysiloxanes would not be suitable for use as an injectable, in situ curable, accommodating IOL. The described polysiloxanes have a high modulus, which would prevent the ciliary muscles and zonules from modifying the shape of a lens refilled with these materials.

US 2005/0070626 describes deformable IOLs having a high RI that are composed of a silicone polymer and a silica reinforcer. The silicone polymer is a polysiloxane having aryl group substituents. However, this material would not be suitable for use as an injectable, in situ curable, accommodating IOL. The methods for synthesising the polysiloxanes described in US 2005/0070626 require the materials to be heated to 100° C. This treatment would cause any polymerisable groups to polymerise and so would result in curing before the material was injected into the capsular bag. Further, the methods of synthesis taught would not produce sufficiently homogenous materials to be suitable for curing in situ. In addition, the material is further unsuitable for in situ curing as it uses hydrosilylation reactions in order to crosslink the macromonomer. Hydrosilylation reactions are known to be exothermic and therefore may damage the surrounding biological tissue if conducted in situ. In addition, the cure process is not a ‘cure on demand’ process; it requires the mixing of two components and then waiting for the reaction to take place. As such the surgeon has a limited timeframe in which to inject the mixture into the capsular bag and make any adjustments to ensure the correct level of refilling has been achieved.

Another potential disadvantage associated with the teaching in WO 03/040154 and US 2005/0070626 is that some of the silane groups react to form SiOH groups. These SiOH groups may then react to form further crosslinking between the macromonomers. This additional crosslinking is of particular concern in applications where the viscosity of the macromonomers and the modulus of any cured polymers are important.

Therefore, it is desirable to formulate an injectable, in situ curable, accommodating lens forming material from polysiloxanes that has a suitable refractive index and the desired mechanical and optical qualities so as to constitute an optimal replacement for the natural lens. It is further desirable to formulate such a material so that the refractive index of the material is adjustable or tuneable so that refractive errors, such as myopia or hyperopia, may be corrected.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

As used herein, the term “comprise” and variations of the term, such as “comprising”, “comprises” and “comprised”, are not intended to exclude other additives, components, integers or steps.

### SUMMARY OF THE INVENTION

When conducting experiments to replace the natural lens with a soft gel, it was surprisingly found that in primates (rhesus) the replacement induced a refractive error in all animals (hyperopia). Similar results were obtained for experiments conducted with ex vivo human eyes. It was expected that if you replace the contents of the natural lens with a polymer of the same refractive index (RI) no refractive error would be induced. Conventional optical modelling suggests that the ‘text book’ average RI of the natural human lens is between 1.40 and 1.41. In particular, a refractive index value of 1.407 has been used. Polydimethylsiloxanes having an RI of 1.407 have been produced.

It has now been shown that the original optical power of a lens can be maintained by refilling the lens with a material having an RI of between 1.421 and 1.446. Generally, the RI of a polysiloxane can be raised or lowered by changing the substituents along the polymer backbone. As a matter of theory, the RI of a siloxane polymer can be raised by:
increasing phenyl/aromatic ring content;
increasing halogen (Br, I, Cl) content;
increasing sulphur content; and/or
reducing the fluorinated content of the polymer,
   and generally lowered by:
increasing the fluorinated content of the polymer;
decreasing phenyl/aromatic ring content;
decreasing halogen (Br, I, Cl) content; and/or
decreasing sulphur content.

However, the molar percentages of various substituents cannot simply be increased or decreased as a matter of course. For example, siloxanes containing high molar percentages of phenyl substitution, which would be required to create high RI materials, suffer from a tendency to solidify. Solidification compromises the properties of the polysiloxanes, rendering them unsuitable for use as injectable, in situ curable, accommodating IOLs. Therefore, this tendency limits the degree of phenyl substitution possible on siloxanes and consequently the resulting RI that can be achieved.

Accordingly, there is also a need for polysiloxanes suitable for use in injectable, in situ curable, accommodating IOLs with a higher RI.

Consequently, in a first aspect the present invention provides a macromonomer of the formula 1:

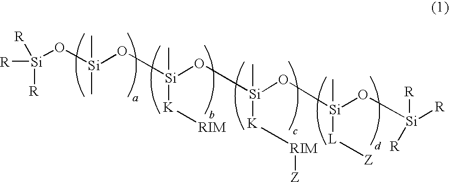

wherein
RIM is a refractive index modifying group;
Z is a free radically polymerisable group;
K is a spacer group;
L is optional and is a spacer group;
each R is independently selected from an RIM, a lower alkyl group, hydrogen or Z;
a is a molar percentage of the macromonomer which is in the range of from 0 to 95 mol %;
b is a molar percentage of the macromonomer which is in the range of from 5 to 99 mol %;
c is a molar percentage of the macromonomer which is in the range of from 0 to 2 mol %; and
d is a molar percentage of the macromonomer which is in the range of from 0 to 2 mol %;
with the proviso that c and d are not both 0 mol %.

In different embodiments the macromonomer has one or more of the following characteristics:
a molecular weight in the range of from 20,000 to 400,000, preferably in the range of from 40,000 to 200,000, and more preferably in the range of from 50,000 to 100,000;
a refractive index at 37° C. in the range of from 1.33 to 1.60, preferably in the range of from 1.41 to 1.5, more preferably in the range of from 1.421 to 1.444, and most preferably in the range of from 1.426 to 1.440;
on average, 1 Z group per 300 or more siloxane repeat units, and more preferably 1 Z group per 550 or more siloxane repeat units;
a viscosity at 25° C. less than 150,000 cSt, preferably less than 80,000 cSt and more preferably in the range of from 1,000 cSt to 60,000; and
when cured into an IOL polymer, a modulus at 37° C. less than 50 kPa, preferably less than 10 kPa and more preferably less than 5 kPa.

Each RIM may independently be any group capable of modifying the RI of the macromonomer. For instance, modification may be a change from the RI of an equivalent polydimethylsiloxane macromonomer. An RIM may modify the RI of the macromonomer by increasing or decreasing the RI. Groups with higher electron density have a tendency to increase the RI of the macromonomer, while groups with a lower electron density have a tendency to reduce the RI or the macromonomer.

The RIM may be a substituted or unsubstituted aromatic group, a fluorinated group, a group containing bromine, iodine, or chlorine atom(s) or a sulphur containing group. Use of substituted or unsubstituted aromatic groups, sulphur containing groups or bromine, iodine or chlorine containing groups will result in a siloxane polymer with an increased refractive index. Alternatively, use of a fluorinated group will lower the refractive index of the siloxane polymer.

The substituted or unsubstituted aromatic group may be a phenyl ring. In addition, an analogous aromatic group to the phenyl ring may be used, such as a fused aromatic derivative, such as naphthalene, anthracene, 1H-phenalene etc, or clusters of aromatic rings attached to a central carbon or silicon atom. The aromatic group may be substituted by one or more substituents including alcohol, chlorine, bromine, iodine, amine, lower alkyl, lower alkenyl and lower alkoxy. Preferably, the substituted or unsubstituted aromatic group is a phenyl ring. Preferably, the substituted phenyl group is not styrene.

Suitable fluorinated groups include perfluorinated C₁ to C₁₂ alkyl. For example, a partly or wholly fluorinated C₄-C₈-cycloalkyl or a group of the following formula:

—[(CH₂)ₐ—(Y)_{z}—(CHF)_{b}—(CF₂)_{c}]—R₂

wherein R₂ is hydrogen or fluorine, Y is a group —N(R₃)SO₂—, —OSO₂—, —OC(O)— or —N(R₃)C(O)—, R₃ is hydrogen or C₁-C₄-alkyl, z is an integer of 0 or 1, a is an integer from 1 to 15, b is an integer from 0 to 6, and c is an integer from 1 to 20.

Sulphur containing groups include thioester or thioether moieties. For example, groups of the following formulas:

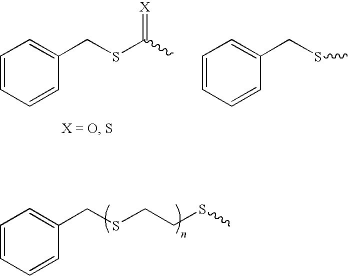

The RIM is preferably a phenyl group, which may be substituted or unsubstituted as described above.

Each Z may independently be any free radically polymerisable group capable of cross-linking the macromonomers to form a polymer in vivo. Preferably, Z is an ethylenically unsaturated group. Suitable groups include acrylate, methacrylate, alkyl methacrylate, acrylamide, methacrylamide, vinyl, styrene, acrylamidoalkyl, methacrylamidoalkyl, acryloxyalkyl and methacryloxyalkyl. Further, suitable precursors for free radically polymerisable groups may be azlactones, isocyanatoethylmethacrylate (IEM), acryloyl chloride, methacrylic anhydride or methacryloyl chloride, particularly when the siloxane macromonomer or siloxane reagent has a pendent alcohol, thiol or amino group.

Each K may independently be any biologically acceptable group capable of linking the refractive index modifying group to the siloxane backbone. K may be a linear, branched, or cyclic lower alkyl, which is optionally interrupted by one or more heteroatoms, such as O, N or S, or functional groups such as, but not limited to, ester, amide, urethane, carbonate, thioester or —C(S)—NH—. Further the lower alkyl may be substituted by a functional group such as, but not limited to, ester, amide, urethane, carbonate, thioester, thiol, alcohol or amine.

Preferably, when K is a linear, branched, or cyclic lower alkyl, K bonds to the silicon atom of the siloxane group via a carbon atom.

Preferably K is a lower alkyl of the formula —(CH₂)ₙ— wherein n is an integer 1, 2, 3, 4 or 5. More preferably n is an integer 2 or 3.

Each L, when present, may independently be any biologically acceptable group capable of linking the free radically polymerisable group above to the siloxane backbone. L may be a linear, branched, or cyclic lower alkyl, which is optionally interrupted by at least one heteroatom, such as O, N or S, or functional group such as, but not limited to, ester, amide, urethane, carbonate, thioester or —C(S)—NH—. Further the lower alkyl may be substituted by a functional group such as, but not limited to, ester, amide, urethane, carbonate, thioester, thiol, alcohol or amine.

Preferably L is a lower alkyl of the formula —(CH₂)ₙ— wherein n is an integer 1, 2, 3, 4 or 5. More preferably n is an integer 2 or 3.

Suitable precursors for L include allyl alcohol, allyl amine, propylene alcohol and allyl cyclohexanol.

Lower alkyl has, in particular, up to 10 carbon atoms, preferably up to 4 carbon atoms which may be straight chain or branched. Such groups for example, include methyl, ethyl, propyl, butyl and pentyl groups.

Lower alkenyl has, in particular, up to 10 carbon atoms, preferably up to 4 carbon atoms which may be straight chain or branched. Such groups for example, include vinyl, allyl and propenyl groups.

a is preferably in the range of from 10 to 88 mol % and more preferably in the range of from 50 to 85 mol %.

b is preferably in the range of from 5 to 70 mol %, more preferably in the range of from 7 to 50 mol % and most preferably in the range of from 10 to 30 mol %.

c is preferably in the range of from 0 to 1.5 mol % and more preferably in the range of from 0 to 1 mol %.

d is preferably in the range of from 0 to 1.5 mol % and more preferably in the range of from 0 to 1 mol %.

In one form of the invention, R is independently selected from RIM and lower alkyl.

In forming the ends of the macromonomer, any reagents capable of forming end groups may be used. The end groups may include free radical polymerisable groups to increase the potential degree of cross-linking of the macromonomer when cured. Suitable reagents for introducing end groups include hexamethyldisiloxane, hexaethyldisiloxane, tetramethyldisiloxane, 1,3-bis(3-aminopropyl)-1,1,3,3-tetramethyldisiloxane, 1,3-bis(3-methacryloxypropyl)tetramethyldisiloxane, 1,3-bis(3-chloropropyl)-1,1,3,3-tetramethyldisiloxane, 1,3-bis(4-hydroxypropyl)-1,1,3,3-tetramethyldisiloxane, 1,1,3,3-tetramethyl-1,3-diphenyldisiloxane and divinyltetramethyldisiloxane.

As will be appreciated, in the formula 1, the RIM, Z, K, L and R groups may vary with the alternatives given in the above description. For example, as one skilled in the art would appreciate, the macromonomer may be synthesised by substituting two or more different -K-RIM, -K-RIM-Z or -L-Z groups onto the siloxane backbone. Accordingly, the invention does not require that every RIM, Z, K, L and R group be identical in a given macromonomer.

The macromonomer may optionally be further substituted with groups having pharmaceutical activity or being capable of acting as UV or blue light filters, polymerisation initiators, such as photoinitiators, thermal initiators or redox initiators or biologically inert capping groups. Substitution with such groups, or other suitable groups, would impart these activities to the resultant polymer. The groups may be incorporated into the macromonomer by a direct bond to a silicon atom or by linking through the -L-Z, -K-RIM-Z or -K-RIM groups or via other suitable methods.

In another aspect, the present invention provides a composition curable into a biomedical device including a macromonomer as described above. The biomedical device is preferably an ophthalmic device. The ophthalmic device may be an IOL, corneal inlay, corneal onlay, contact lens, or an artificial cornea. Preferably the device is an IOL. More preferably, the device is an injectable, in situ curable, accommodating IOL. Accordingly, a preferred embodiment of the present invention is a composition curable in situ to form an accommodating IOL including a macromonomer as described above. A further preferred embodiment is an injectable, in situ curable IOL composition including the macromonomer described above.

The composition can be injected into the lens capsular bag and then cured in situ, for example, by visible or ultra violet light. The lens once formed has a sufficiently low modulus that the ciliary muscles controlling the zonules can adjust the lens shape in the usual way, thus enabling the lens to accommodate.

The present invention also encompasses the use of the above composition as a biomedical device, preferably an injectable, in situ curable, accommodating IOL.

In a further aspect, the present invention provides biomedical devices, preferably accommodating IOLs, formed from the above composition.

Advantageously, macromonomers of the present invention allow the RI of the material to be tailored to the particular application required. Typically the RI will be higher than that normally measured for the natural lens which the IOL is replacing. The IOL may replace the natural lens, or a previously implanted IOL in the eye. The RI of the IOL is adjusted or “tuned’ to that required for treating the eye by altering the molar percentage of RIM groups in the macromonomer. Desirably the IOL formed from the composition has similar physical characteristics to a healthy natural lens, particularly elasticity. The macromonomers also preferably have a viscosity before curing that permits injection of the macromonomers into a capsular bag. The viscosity is preferably less than 150 000 cSt, more preferably less than 80 000 cSt.

In another aspect the present invention provides a method of implanting an IOL including introducing a composition as described above into a lens capsular bag and then curing the composition. The present invention also includes methods of treating a refractive error including implanting an IOL as described above.

In one aspect, the invention includes the use of the composition in the manufacture of an accommodating IOL for correcting refractive error in an eye, or maintaining the refractive power of an eye. The invention further extends to an eye having an IOL formed from a composition as described above.

The invention also extends to a method of forming a medical device or prosthesis, including an IOL, with a refractive index of more than 1.33 by polymerising macromonomers as described above. Preferably the polymerisation is conducted in situ.

### BRIEF DESCRIPTION OF THE DRAWINGS

FIG. 1 is a plot of refractive index at 37° C. against the concentration of tetramethyltetrapropylbenzene cyclotetrasiloxane in mol % in the reaction feed.

FIG. 2 is a plot of refractive index at 37° C. against the concentration of tetramethyltetrapropylbenzene cyclotetrasiloxane in mol % in the reaction feed for a greater concentration range than FIG. 1.

FIG. 3 is a plot of the molar ratio of tetramethyltetrapropylbenzene cyclotetrasiloxane in feed against the molar ratio of methylpropylbenzene siloxane units in the resulting macromonomer (as determined by NMR analysis) providing a calibration curve for determining synthesis parameters.

### DETAILED DESCRIPTION OF THE EMBODIMENTS

The macromonomers of the present invention offer the advantage that they may not only form high refractive index polymers but also exhibit desired mechanical and chemical characteristics, particularly when used as injectable precursors for an accommodating IOL. Furthermore, the refractive index of the macromonomers may be controlled during synthesis to enable preparation of a range of polymers having various refractive indices.

The macromonomers of the present invention which are described above may be random or block type macromonomers. Typically, the macromonomers are random macromonomers.

Macromonomers of the present invention may have a molecular weight in the range of from 20,000 to 400,000, preferably in the range of from 40,000 to 200,000, and more preferably in the range of from 50,000 to 100,000.

The macromonomers of the present invention may be synthesised by any suitable method known in the art.

An advantageous method by which refractive index modifying groups and/or polymerisable group may be attached to a siloxane macromonomer is to use a hydrosilylation reaction. For instance, using hydrosilylation, free radically polymerisable groups and refractive index modifying groups are attached to the siloxane backbone using allyl-precursors in methods known to those skilled in the art. For example, phenyl functionalized allyl-precursor or the like include allyl benzene, styrene, allyl phenol, allyl phenoxy and eugenol and free radical polymerisable functionalized allyl-precursors or the like include allyl (meth)acrylate and allyl isocyanate. Scheme 1 illustrates a hydrosilylation reaction and suitable reagents containing phenyl groups.

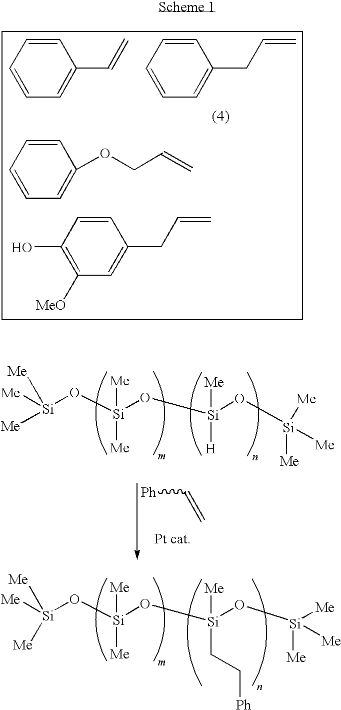

The addition of refractive index modifying groups and free radically polymerisable groups using hydrosilylation reactions may be either to macromonomers, which are silane functionalized, or to silane functionalized cyclic siloxane intermediates before they are subjected to ring opening polymerisation to form the macromonomer. Suitable cyclic siloxane intermediates for functionalisation using this approach include tetramethylcyclotetrasiloxane (D₄^{H}), trimethylcyclotrisiloxane (D₃^{H}), pentamethylcyclopentasiloxane (D₅^{H}) or hexamethyl-cyclohexasiloxane (D₆^{H}).

The following description and schemes describe various approaches to substituting free radically polymerisable groups and refractive index modifying groups, although the examples relate specifically to phenyl containing refractive index modifying groups, through hydrosilylation reactions.

In the schemes, where figures such as “a=80, b=20” are provided, these are mol % values for the various substituents indicated. In the schemes, a, b, c and d etc do not necessarily directly correspond to the integers a, b, c and d as defined for Formula 1. Moreover, in the schemes, where a proportion a, b, c etc of a macromonomer is reacted, the use of the same letter in the reaction product macromonomer does not necessarily imply that the reaction proceeded to 100% completion. Therefore, through the reactions illustrated, there will inevitably be some change in the relative proportions of the substituted siloxane backbone components.

One approach is to prepare silane functionalised macromonomer with sufficient silane functionality to allow introduction of both the phenyl groups and polymerisable groups.

For instance, the silane functionalised macromonomer is sequentially functionalized as depicted in scheme 2. For example the silane macromonomer is firstly modified with allyl benzene, isolated, and then functionalized with a second allyl derivative such as allyl alcohol. The introduced alcohol groups are further used to attach polymerisable groups by reacting with a suitable substance containing polymerisable group such as azlactone, isocyanatoethylmethacrylate (IEM), acryloyl chloride or methacryloyl anhydride.

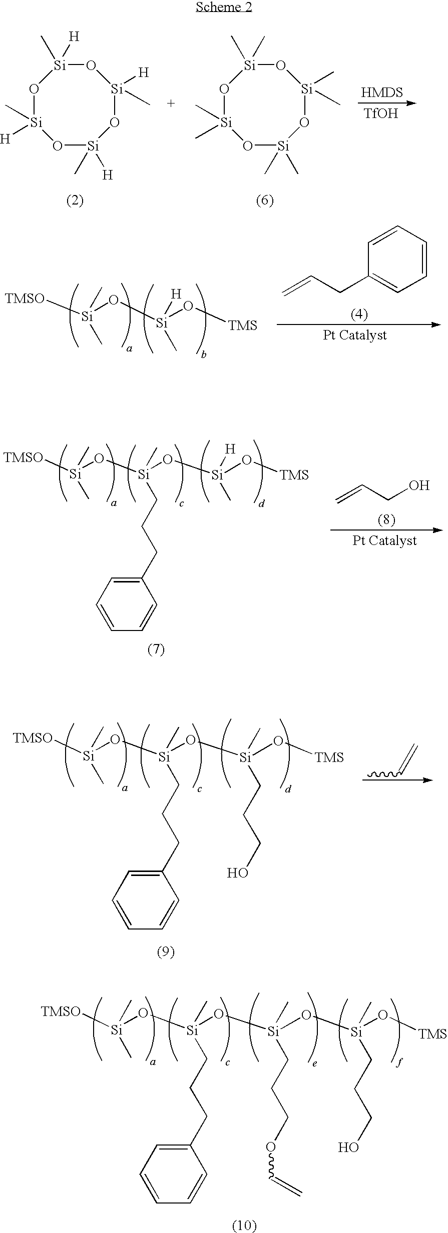

Alternatively, the silane functionalised macromonomer undergoes parallel functionalization as depicted in scheme 3. A mixture of allyl derivatives may be hydrosilylated on to the silane macromonomer in one step. For example, a mixture of eugenol (11) and allyl benzene (4) or eugenol (11) alone is hydrosilylated onto the silane macromonomer (5). The alcohol groups of the eugenol are further used to introduce polymerisable groups by reacting with a suitable substance containing polymerisable group such as azlactone, IEM, acryloyl chloride or methyacryloyl anhydride. Two examples of Z are given as Z¹ and Z².

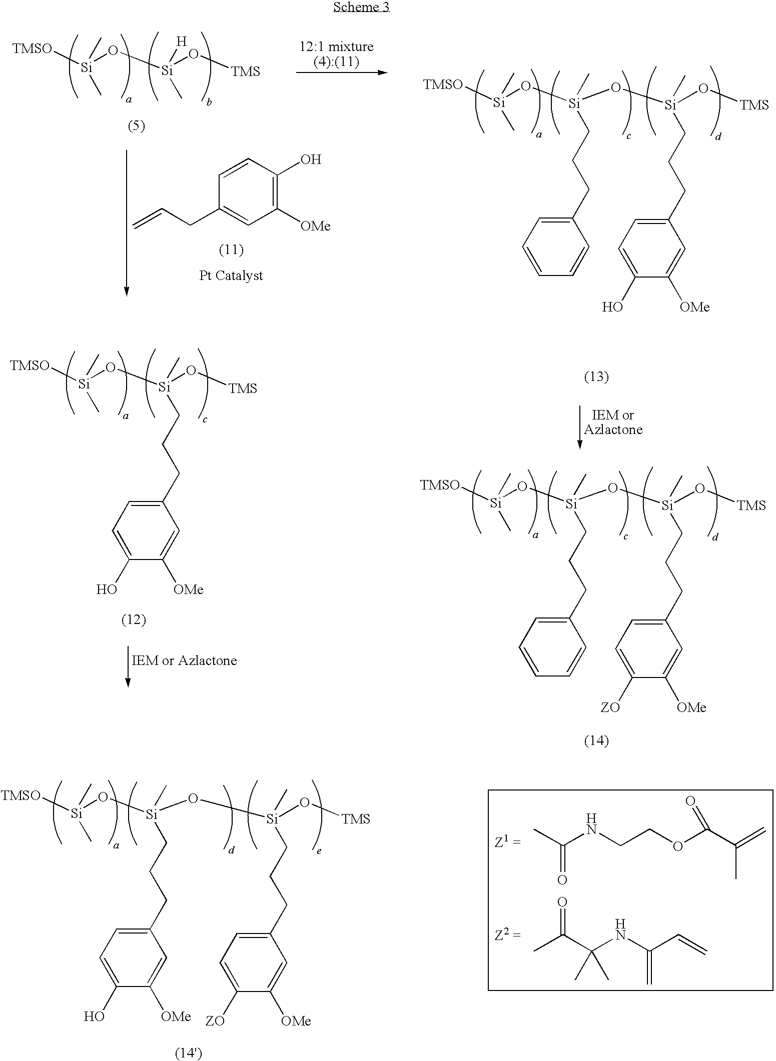

The relative ratio of the hydrosilylated groups are controlled in the product by controlling the feed ratio of the starting components. For example, as shown in Scheme 4, controlling the feed ratio of allyl benzene to eugenol gives macromonomers with predictable and controllable mol % ratios.

Scheme 4

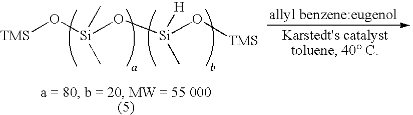

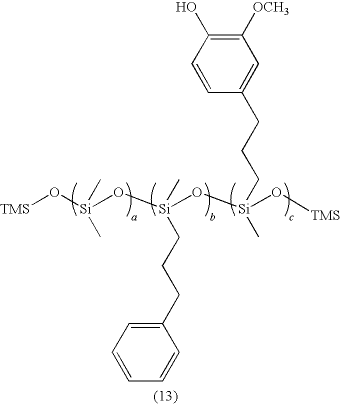

| allyl benzene:eugenol | theoretical composition | MW of resultant |
|---|---|---|
| free ratio | mol % | macromonomer |
| 100:1 | a = 80 b = 19.80 c = 0.20 | 55 000 |
| 50:1 | a = 80 b = 19.60 c = 0.40 | 55 000 |
| 25:1 | a = 80 b = 19.20 c = 0.80 | 55 000 |

Instead of parallel functionalization with mixtures of similar phenyl functionalised allyl derivatives, parallel functionalization can also take place between dissimilar allyl derivatives, for example allyl alcohol and allyl benzene as shown in Scheme 5. The alcohol groups are then modified to introduce polymerisable groups (eg by reacting with azlactone, IEM, acryloyl chloride or methyacryloyl anhydride).

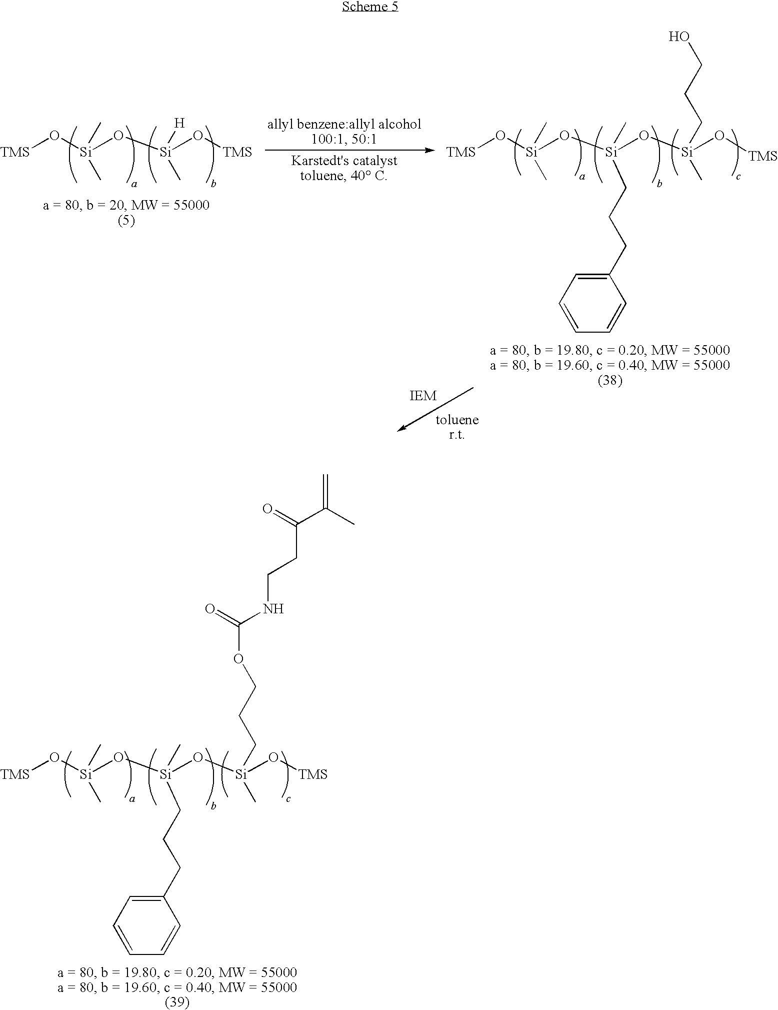

The pendent alcohol functional groups may react with a substance containing polymerisable groups as described above. Alternatively they can be capped with inert groups, for example as depicted in Scheme 6. Capping a portion of the pendent alcohol groups with inert groups assists in further controlling the crosslinking density of the final cured polymer, by reducing the number of free radically polymerisable groups that are introduced.

Furthermore, in some biological applications it is advantageous to cap any remaining free hydroxyl groups with inert groups so as to minimise any potentially disadvantageous interactions when in vivo. Alternatively, such hydroxyl groups are useful sites for binding other biologically active components, such as drugs, UV filters and other appropriate molecules, as described above.

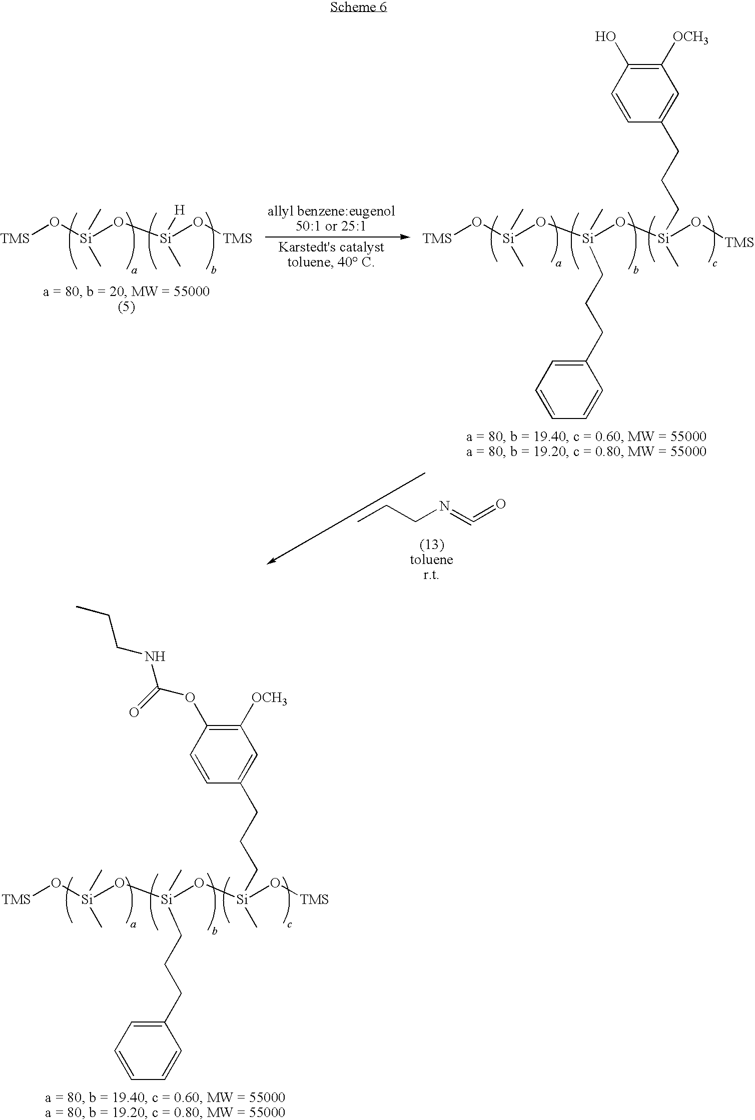

In a further alternative method of introducing phenyl and polymerisable groups to a silane functionalised macromonomer, the introduction of polymerisable groups is performed in one step along with the introduction of the phenyl groups. Such a method is depicted in Scheme 7 where a Eugenol-IEM adduct is added to the hydrosilylation mixture to introduce the polymerisable groups.

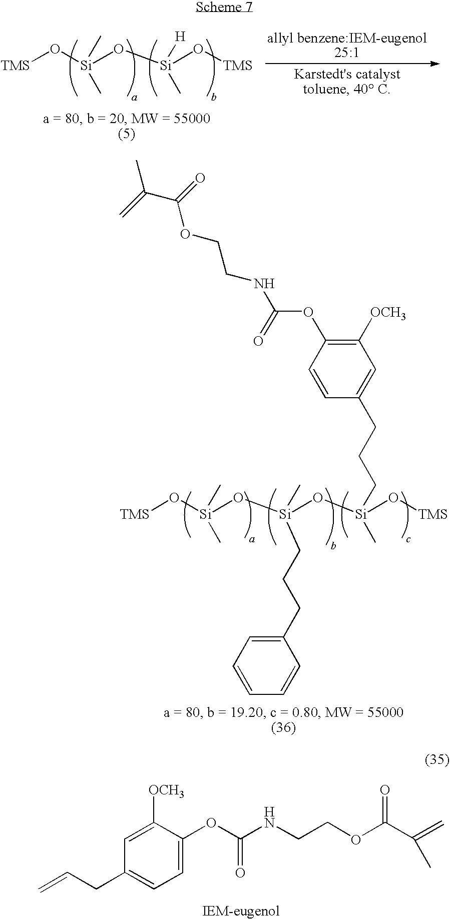

In an alternative to functionalising a silane functionalised macromonomer aforementioned, a cyclic intermediate monomer may be first functionalised with phenyl or polymerizable groups and then subjected to ring opening polymerisation. In a preferred method trimethylcyclotrisiloxane or tetramethylcyclotetrasiloxane (often also referred to as D₃H or D₄H) or a similar silane functionalised cyclosiloxane, (e.g. D₅H and D₆H) is firstly functionalized with phenyl rings and/or polymerisable groups. Then the functionalized cyclosiloxanes are ring opened to obtain the desired macromonomer containing both RI modifying and polymerizable groups.

An example of this is scheme 8 which shows the synthesis of eugenol functionalised D₄ (D₄^{E}). D₄^{E} is then ring opened in the presence of octamethylcyclotetrasiloxane (D₄), allyl benzene functionalized tetramethylcyclotetrasiloxane (D₄^{AB}), and end group hexamethyldisiloxane (HMDS) to give the premacromonomer (20). Polymerisable groups are attached to the alcohol groups of the eugenol by reacting with suitable polymerisable molecules (eg azlactone, IEM, acryloyl chloride or methyacryloyl anhydride). Two examples of Z are given as Z¹ and Z².

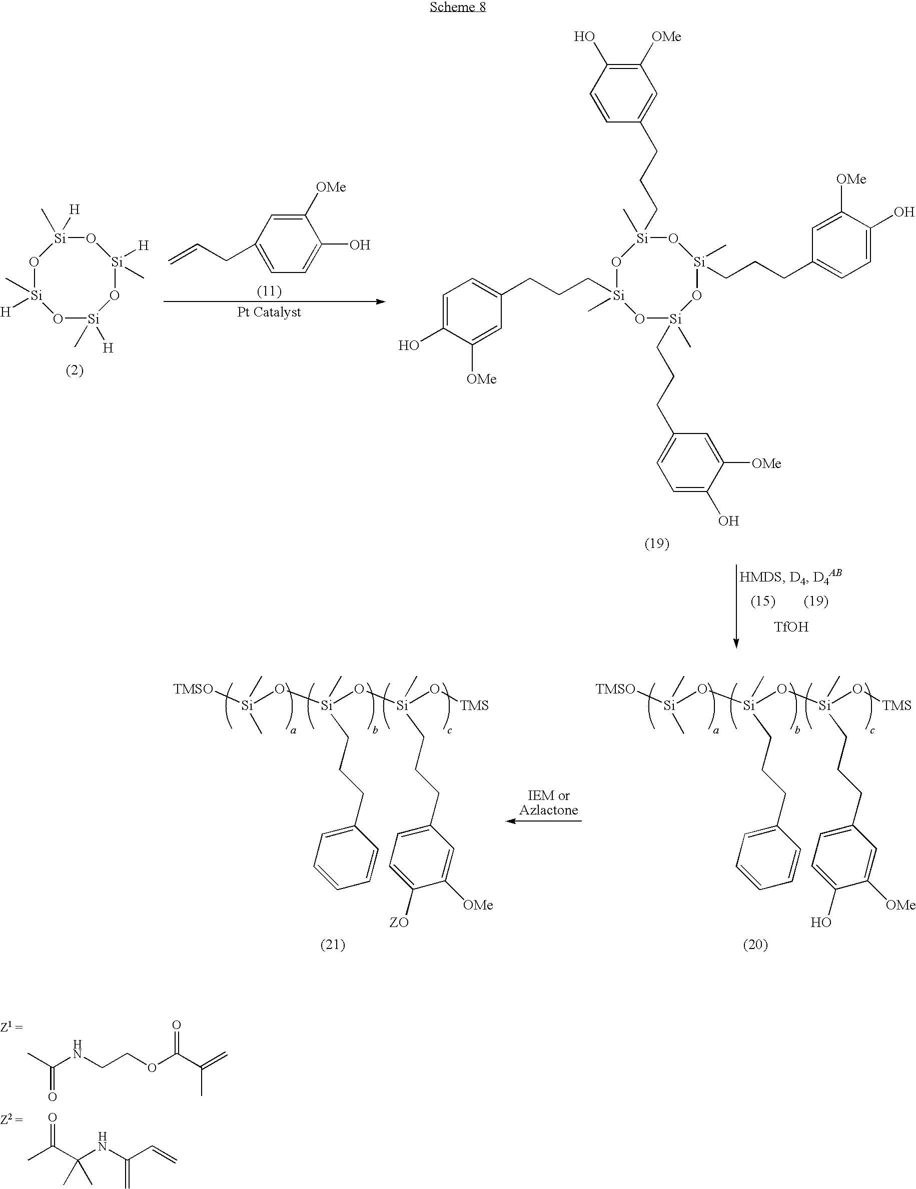

A variety of phenyl functionalised cyclic siloxanes may also be prepared. Scheme 9 shows the synthesis of allyl benzene and allyl methylacrylate functionalised cyclosiloxane (D₄^{AB} and D₄^{AM}, respectively).

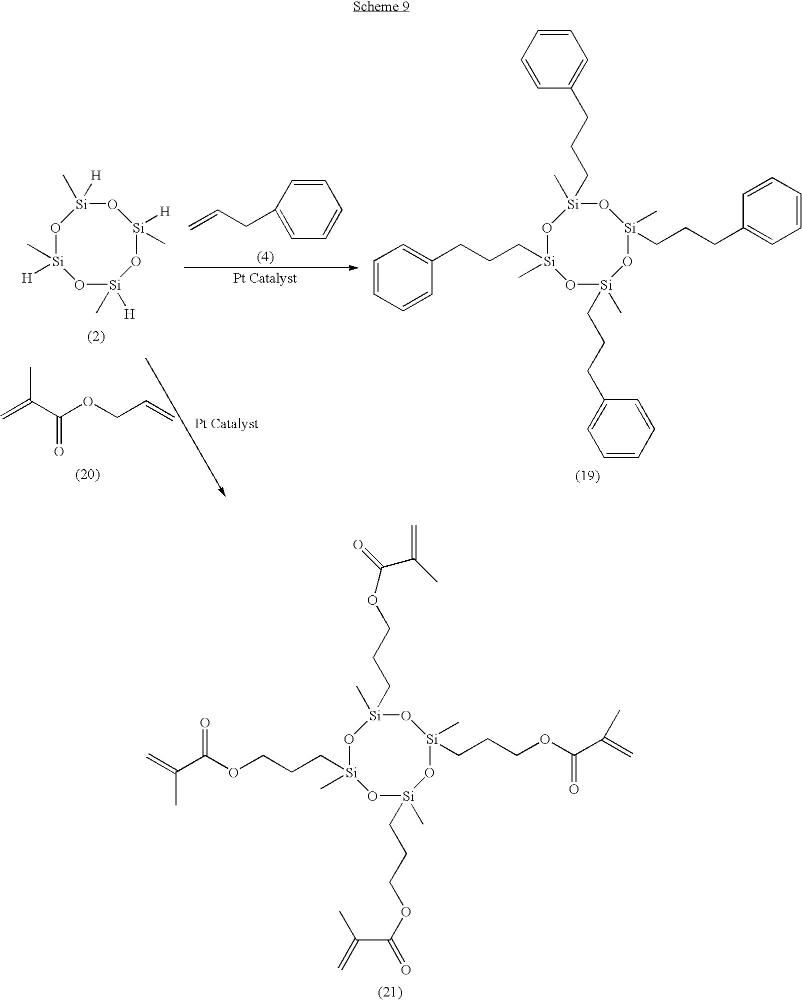

A combination approach may also be used to prepare the desired siloxane polymers. In addition to functionalised cyclosiloxane, D₄^{H} is added to the ring opening mixture, such that phenyl groups are introduced to the macromonomer by ring opening polymerisation and polymerisable groups are introduced by functionalization of silane groups in the macromonomer as shown in Scheme 10. Again similar to the above routes, the polymerisable groups are introduced in one or multiple steps. Two examples of Z are given as Z¹ and Z².

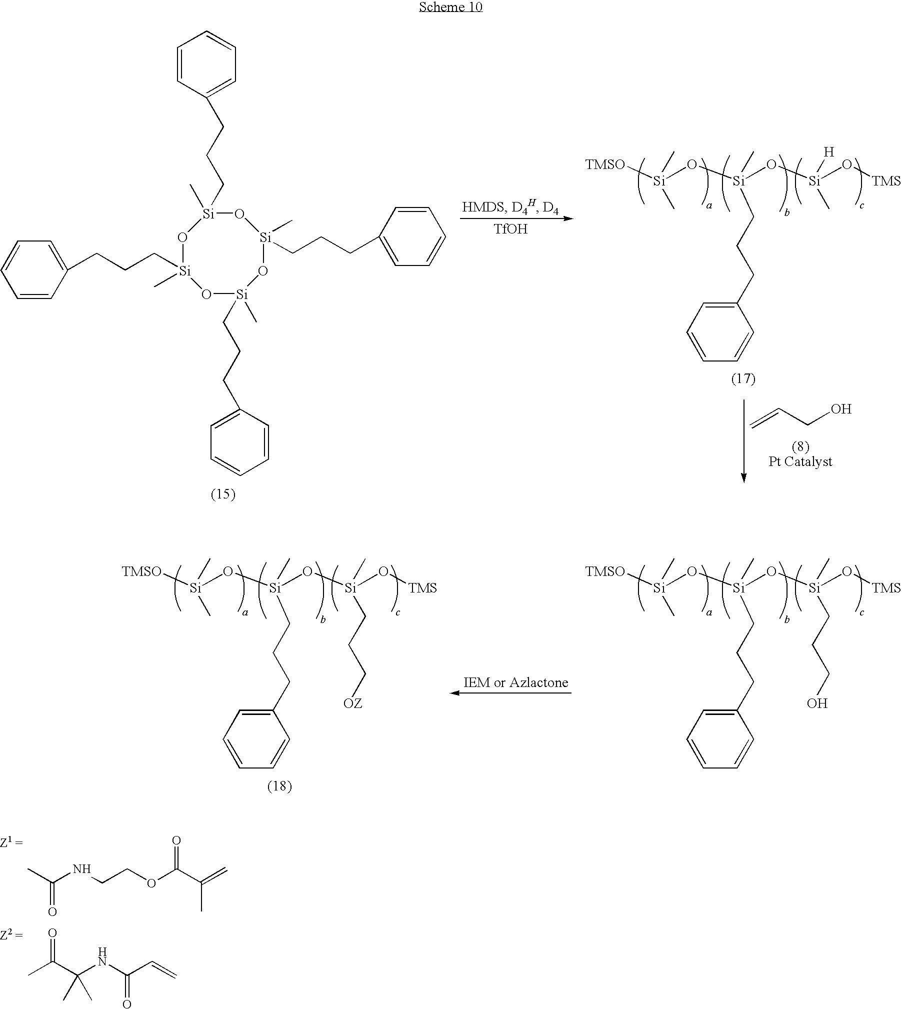

Alternatively, the introduction of phenyl and polymerisable groups to the macromonomers is performed in one step by ring opening a phenyl functionalised cyclosiloxane and a polymerisable group functionalised cyclosiloxane in a mixture with an end group blocker, eg divinyltetramethyldisiloxane (DVTMDS), as shown in Scheme 11. Advantageously, the ratios of the components in the final product are able to be controlled by controlling the feed ratio of the components in the ring opening polymerisation step.

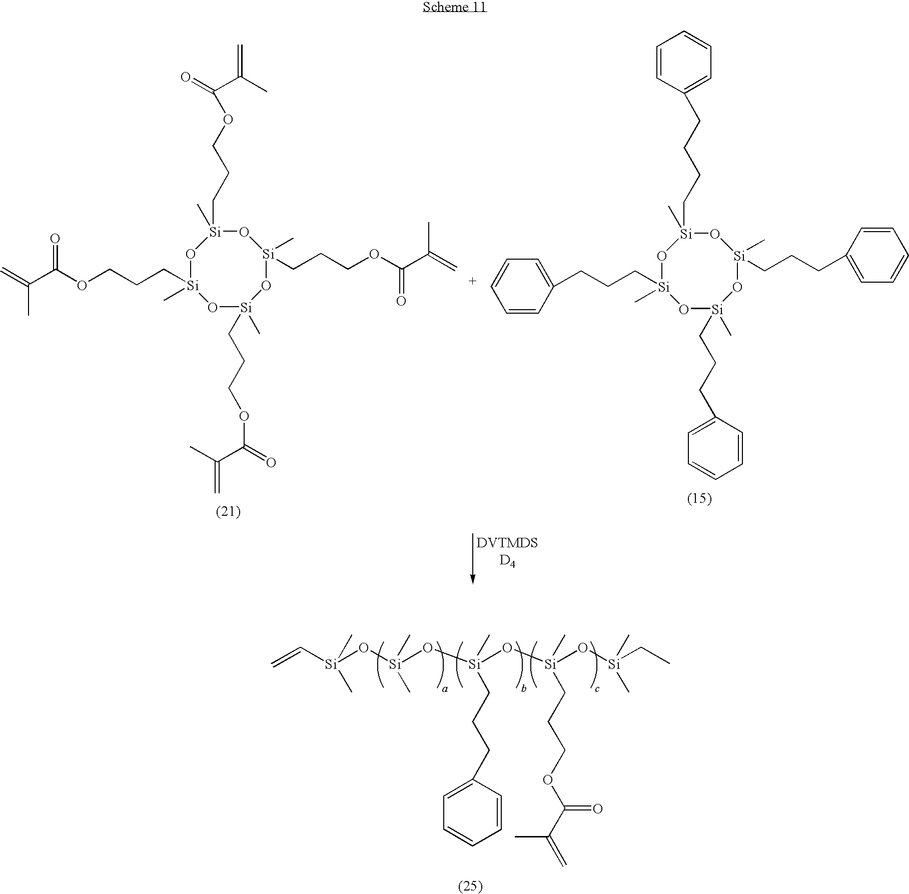

Scheme 12 illustrates another example of a ‘one step’ synthesis. IEM-eugenol adduct (26) is first prepared then reacted with D₄^{H}. The IEM-eugenol D₄^{H} derivative is then ring opened with D₄^{AB}, D₄ and end group blocker DVTMDS to produce a polymerisable siloxane macromonomer of high refractive index.

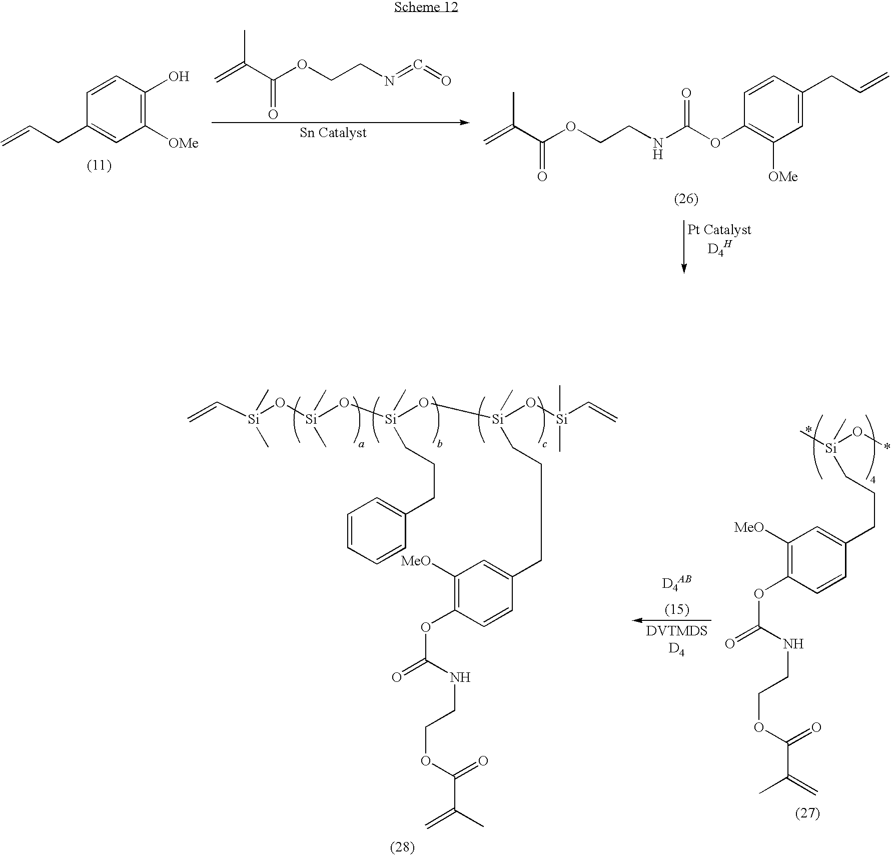

Scheme 13 shows a ‘two step’ synthesis. Another D₄^{H} phenyl derivative is first prepared by hydrosilylation of allyl phenol with D₄^{H} with allyl phenol. The functionalized cyclosiloxane (31) is then ring opened with D₄^{AB}, D₄ and an end group. The phenolic hydroxyls are capped with IEM to afford a polymerisable siloxane of high refractive index.

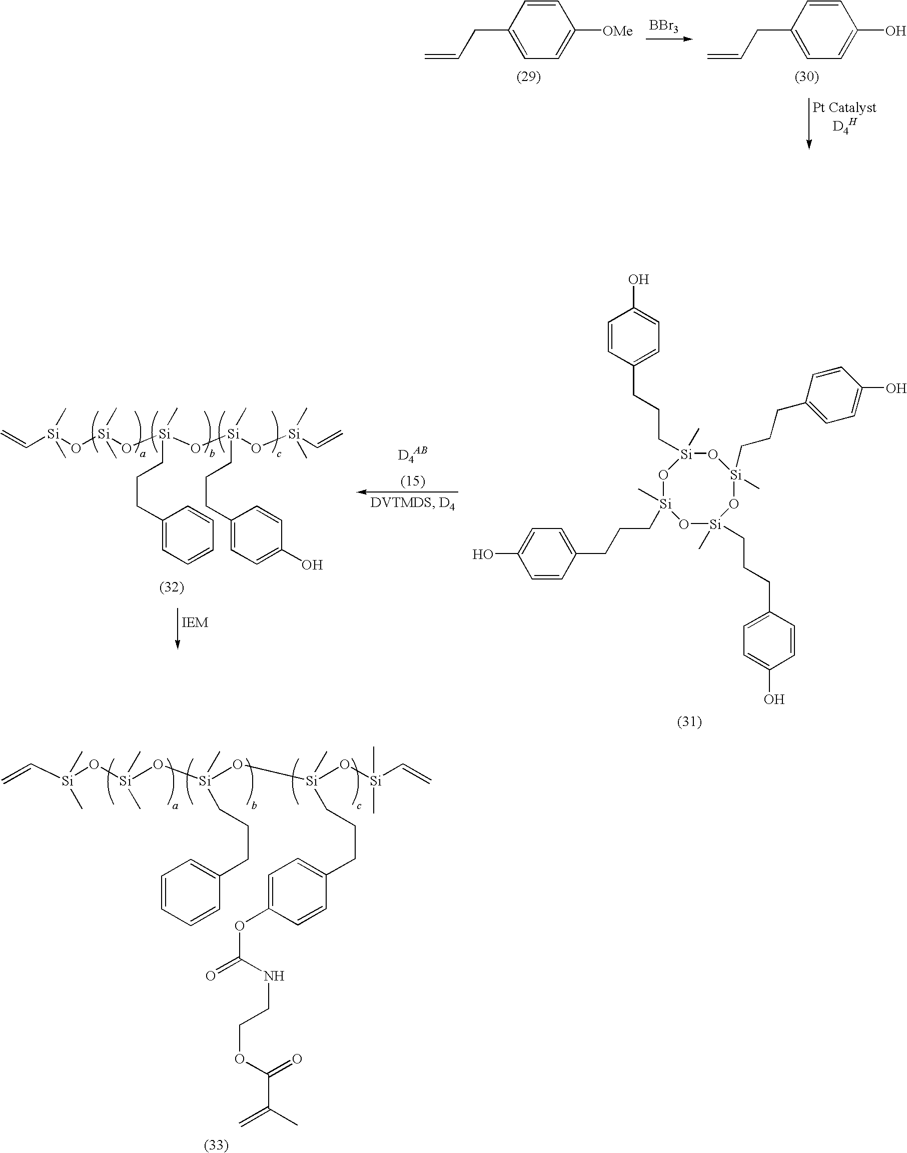

In an alternative method, a cyclic intermediate monomer functionalised with only one refractive index modifying group (RIM) or polymerisable group (Z) (monofunctionalised cyclosiloxane) may be formed and then subjected to ring opening polymerisation. In a preferred method dichloromethylsilane is functionalised with a refractive index modifying group (eg phenyl or fluoroalkyl group) or a polymerisable group. The resulting compound is then reacted with a 1,3-dihydroxytetramethyl-disiloxane to form a monofunctionalised pentamethylcyclotrisiloxane. Alternatively, 1,3-dihydroxytetramethyldisiloxane is reacted with dichloromethylsilane to form pentamethylcyclotrisiloxane, which is subsequently functionalised with a phenyl or polymerisable group. Alternatively monofunctional cyclotetrasiloxanes may be prepared by using 1,3-dihydroxyhexamethyltrisiloxane instead of 1,3-dihydroxytetramethyl-disiloxane in the above reaction scheme. In addition, difunctional derivatives may be prepared by using dichlorosilane instead of dichloromethylsilane. Then the phenyl and polymerisable functionalized cyclosiloxanes are ring opened in the presence of D₄ to obtain the desired macromonomer containing both RI modifying and polymerizable groups. An example of this is scheme 14.

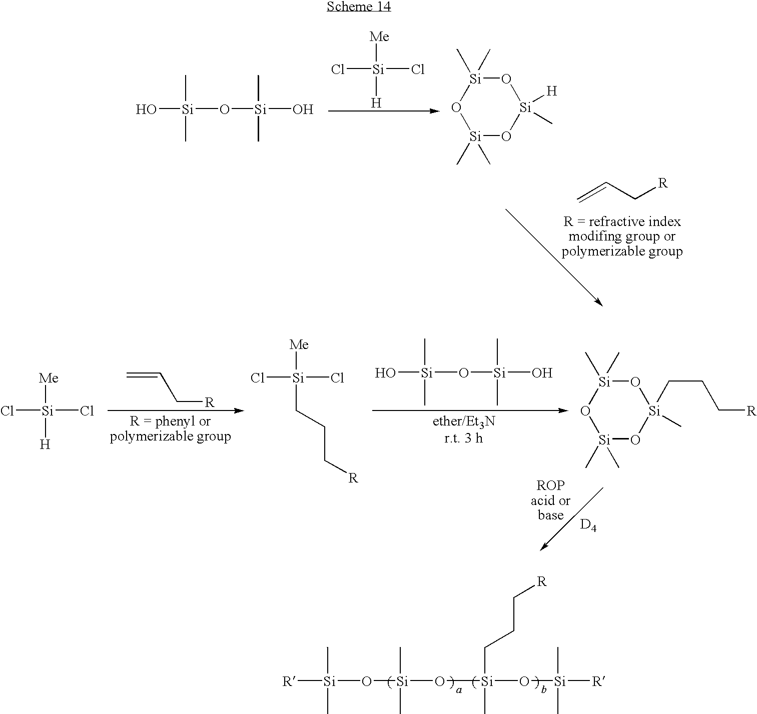

The refractive index of the macromonomer can be tuned to the desired level by adjusting the molar ratio of refractive index modifying group substituents in the macromonomer.

When functionalising a macromonomer having silane groups the relative ratio of the refractive index modifying group reagents and the free radically polymerisable group reagents can be controlled to provide a predictable level of refractive index modifying group substituent in the macromonomer.

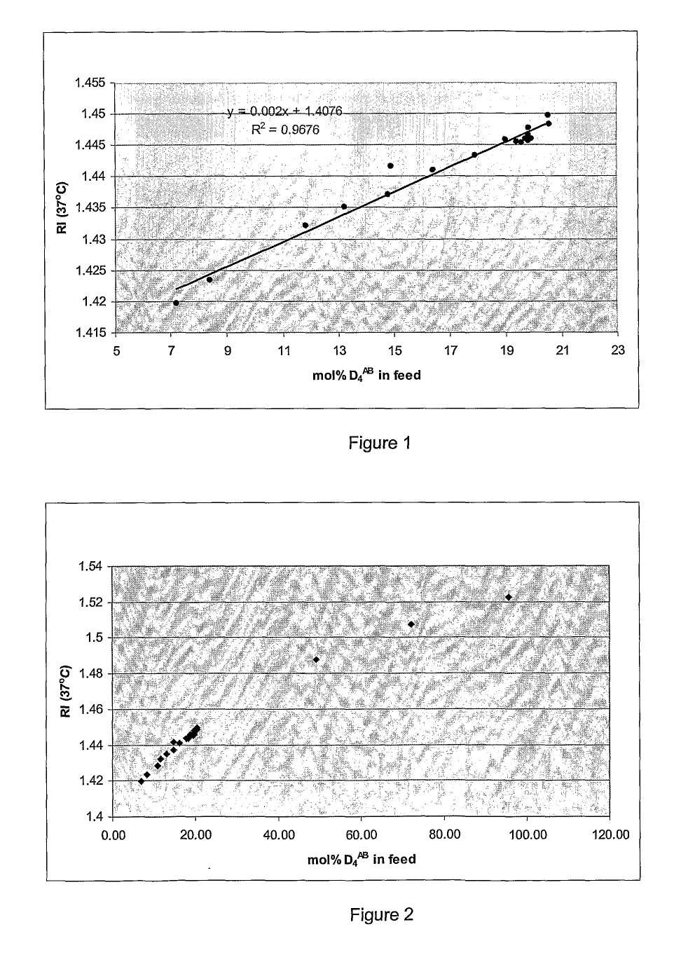

Alternatively, when previously functionalised cyclosiloxanes are used in a ring opening polymerization the refractive index of the macromonomer may be tuned by adjusting the concentration of the refractive index modifying group substituent in the ring opening reaction mixture. FIGS. 1 and 2 show the relationship between the D₄^{AB} molar ratio in the reaction feed and the refractive index of the resultant macromonomer at 37° C. The existence of this relationship allows one manufacturing a biomedical device, such as an IOL, to reliably produce a polymer having a particular desired refractive index. This is particularly advantageous in optical applications.

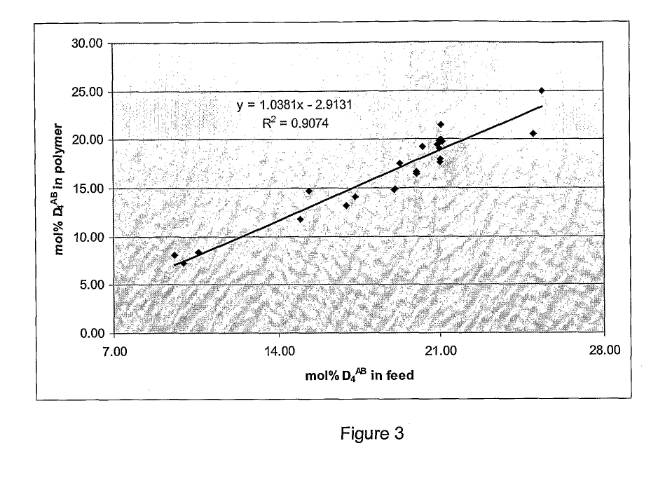

Further, in order to finely control the molar ratio of the refractive index modifying group and thus the refractive index of the macromonomer, efficiency of the ring opening polymerization may be accounted for. FIG. 3 shows a calibration curve between the molar ratio of the refractive index modifying group, in this case D₄^{AB}, in the feed (horizontal axis) and the molar ratio of the refractive index modifying group, D₄^{AB}, in the macromonomer (vertical axis). The molar ratio of refractive index modifying group incorporated in the macromonomer may be determined by NMR analysis.

The macromonomers of the present invention may be cured via free radical polymerisation to form crosslinked polymers. Known curing processes may be used to form the crosslinked polymers.

The crosslinking process is preferably carried out in such a way that the resulting network polymer is free or essentially free of undesired constituents. A particular undesired constituent is starting macromonomers that have had none of their polymerisable groups incorporated into the network and as such are potentially extractable from the resulting network polymer after cure.

In the case of photo cross-linking, it is expedient to add an initiator which is capable of initiating free-radical crosslinking. It is preferred that the initiators are activated by light in the visible spectrum rather than UV range as this enables the use of frequencies to cure the polymer that are not harmful to the eye or retina.

Examples thereof are known to the person skilled in the art; suitable photoinitiators which may be mentioned specifically are benzoins, such as benzoin, benzoin ethers, such as benzoin methyl ether, benzoin ethyl ether, benzoin isopropyl ether and benzoin phenyl ether, and benzoin acetate; acetophenones, such as acetophenone, 2,2-dimethoxyacetophenone and 1,1-dichloroacetophenone; benzil, benzil ketals, such as benzil dimethyl ketal and benzil diethyl ketal, camphorquinone, anthraquinones, such as 2-methylanthraquinone, 2-ethylanthraquinone, 2-tert-butylanthraquinone, 1-chloroanthraquinone and 2-amylanthraquinone; furthermore triphenylphosphine, benzoylphosphine oxides, for example 2, 4,6-trimethylbenzoyl-diphenylphosphine oxide; Eosin homologues such as Eosin Y, Phloxine, Rose Bengal and Erythrosin; benzophenones, such as benzophenone and 4,4′-bis(N,N-dimethylamino)benzophenone; thioxanthones and xanthenes; acridine derivatives; phenazine derivatives; quinoxaline derivatives and 1-phenyl-1,2-propanedione 2-O-benzoyl oxime; 1-aminophenyl ketones and 1-hydroxyphenyl ketones, such as 1-hydroxycyclohexylphenyl ketone, phenyl 1-hydroxyisopropyl ketone, 4-isopropylphenyl 1-hydroxyisopropyl 1-hydroxyisopropyl ketone, 2-hydroxy-1-[4-2(-hydroxyethoxy)phenyl]-2-methylpropan-1-one, 1-phenyl-2-hydroxy-2-methylpropan-1-one, and 2,2-dimethoxy-1,2-diphenylethanone, all of which are known compounds.

Particularly suitable photoinitiators, which are usually used with visible light sources are IRGACURE®819, Eosin homologues such as Rose Bengal, Eosin B, and fluorones such as H-Nu 470, H-Nu635 and derivatives.

Particularly suitable photoinitiators, which are usually used with UV lamps as light sources, are acetophenones, such as 2,2-dialkoxybenzophenones and hydroxyphenyl ketones, in particular the initiators known under the trade names IRGACURE®651 and IRGACURE@184. A particularly preferred photoinitiator is IRGACURE®819. The photoinitiators are added in effective amounts, expediently in amounts from about 0.05 to about 2.0% by weight, in particular from 0.1 to 0.5% by weight, based on the total amount of cross-linkable macromonomer. In addition the photoinitiator can be incorporated/grafted onto the polymer backbone. Such immobilisation of the polymer has the advantage of reducing the availability of photoinitiator residues from extraction post cure.

The resultant cross-linkable macromonomer can be introduced into a mould using methods known per se, such as, in particular, conventional metering, for example drop wise. Alternatively, the macromonomers may be cured in situ, as for example in the case of an injectable IOL. In this case the macromonomer is cured or crosslinked in the lens capsule after injection.

The cross-linkable macromonomers which are suitable in accordance with the invention can be crosslinked by irradiation with ionising or actinic radiation, for example electron beams, X-rays, UV or VIS light, ie electromagnetic radiation or particle radiation having a wavelength in the range from about 280 to 750 nm. Also suitable are UV lamps, He/Dc, argon ion or nitrogen or metal vapour or NdYAG laser beams with multiplied frequency. It is known to the person skilled in the art that each selected light source requires selection and, if necessary, sensitisation of the suitable photoinitiator. It has been recognised that in most cases the depth of penetration of the radiation into the cross-linkable macromonomer and the rate of curing are in direct correlation with the absorption coefficient and concentration of the photoinitiator. Curing might also be achieved by employing one or more of these methods, eg, heat and light.

If desired, the crosslinking can also be initiated thermally. It should be emphasised that the crosslinking can take place in a very short time in accordance with the invention, for example, in less than twelve hours, preferably in less than an hour, more preferably in less than 30 minutes.

In forming the polymer, the macromonomer is preferably used without the addition of a comonomer although a comonomer may be included. While generally the polymers of the present invention do not usually involve the use of other macromonomers, these may be optionally included. Preferably the polymers comprise at least 50%, more preferably at least 80%, by weight of macromonomers of the present invention.

Macromonomers of the present invention may be used to form biomedical devices, preferably ophthalmic devices. Such devices include IOLs, corneal inlays, corneal onlays, contact lenses, and artificial corneas.

In a preferred application, macromonomers of the present invention are used to form injectable, in situ curable, accommodating IOLs. In this application, the mechanical and optical properties of a cured polymer of the macromonomers are preferably selected to match or restore those properties of the natural biological material of the lens.

One relevant mechanical property for IOLs is the flexibility of such a polymer. Suitable flexibility enables the ciliary muscle/ciliary body and zonules of the accommodative apparatus of the eye to modify the shape of a lens filled with the material, thus providing accommodation. Flexibility is measured by its elasticity modulus (E modulus). The polymer shear modulus is a related property that may be measured also. Both can be measured as the force required to deform a product, such as a lens, formed by the polymer by measuring stress against strain. The E modulus of the polymer of the invention may be measured by a Micro Fourier Rheometer. A Bohlin controlled stress rheometer may also be used. For an injectable, in situ curable, accommodating lens application of this invention, the E modulus measured by a Micro Fourier Rheometer is preferably less than 10 kPa and more preferably less than 5 kPa. The E modulus is influenced by the number of polymerisable groups per macromonomer chain, ie crosslink density and also average spacing (ie the relative proportion of the polymerisable group unit) of the polymerisable groups. Generally, as the number of polymerisable groups per macromonomer molecule decreases or the average spacing between polymerisable groups increases (as a function of the monomeric proportions) the elasticity of the cured polymer decreases.

A relevant optical property for an IOL is the RI of the polymer. The RI at 37° C. may be in the range of from greater than 1.33 to 1.60, preferably in the range of from 1.41 to 1.5, more preferably in the range of from 1.421 to 1.444, and most preferably in the range of from 1.426 to 1.440. The RI may be chosen depending on the refractive error being treated by the IOL.

When used as an injectable material the macromonomers should have a viscosity less than 150,000 cSt and more preferably less than 80,000 cSt at 25° C. Instruments such as the Brookfield rheometer or the Bohlin controlled stress rheometer may be conveniently used for viscosity measurements.

It will be appreciated that while the macromonomers of this invention may be used alone to form the lenses and other biocompatible materials, other materials may also be present in compositions used to form the biomedical devices. For example, diluents may be present as well as other monomers, including other macromonomers, as discussed above. Other additives to the macromonomer precursor, which may be free or grafted onto the polymer backbone, can include ultraviolet absorbers and pharmaceutically active compounds, such as those that inhibit or kill the cells associated with PCO (Posterior Capsule Opacification).

When used as an injectable, in situ curable, accommodating IOL, the composition including macromonomers of the invention may be introduced into the lens using an operation that is in many respects identical to a current cataract extraction and IOL implantation technique (e.g. extra-capsular extraction procedure) with some minor differences. Generally, a small corneal incision is made at the para-limbal region to provide access to the anterior segment. Following dilation of the pupil using a pharmacological agent such as atropine or cyclopentolate, a small capsulorhexis (around 1 mm or less in diameter) is made manually at the periphery of the anterior capsule. Through the small corneal incision and peripheral mini-capsulorhexis, the lens core (including the cortex and nucleus) are extracted. The composition including macromonomers of the invention is injected into the intact lens capsule using a fine gauge (e.g. 29-G or finer) cannula and syringe to reform the lens. The composition is then cured, such as by exposure of the eye to visible or ultra violet light.

Following such techniques and by selecting appropriate pharacteristics, such as RI and modulus, IOLs formed from macromonomers of the present invention may be used to treat presbyopia, myopia or hyperopia.

### EXAMPLES

### Example 1

### Preparation of Functional Cyclic Siloxanes by Hydrosilylation of 1,3,5,7-tetramethylcyclotetrasiloxane (D₄^{H})

The product obtained by hydrosilylation reaction is a siloxane compound represented by the following scheme:

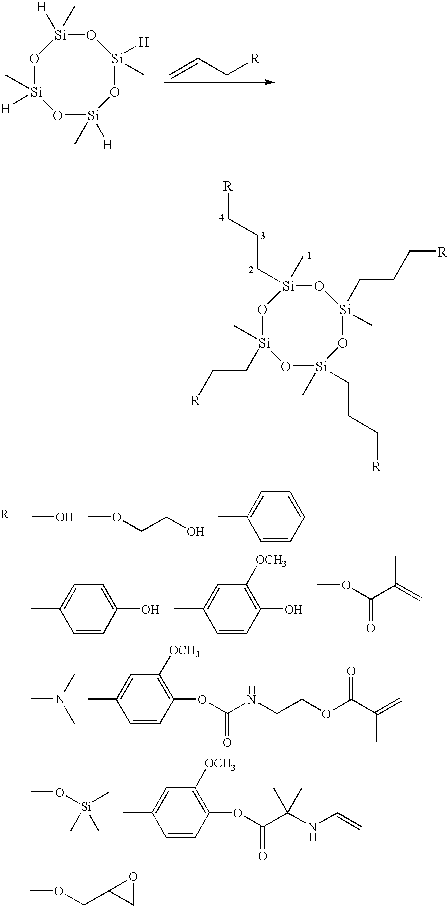

### Example 1A

### Preparation of a cyclotetrasiloxane monomer functionalized by allyl methacrylate (D₄^{AM})

2 g of tetramethylcyclotetrasiloxane (D₄H) was dissolved in 40 ml of dry toluene in a round bottom flask equipped with a reflux condenser. To this solution was added 10 drops (0.180 g) of Karstedt's catalyst ([Pt]=3.4×10⁻⁵ mol/ml). The flask was shrouded in aluminium foil to exclude light. 4.62 g of distilled allyl methacrylate was added dropwise from the top of the condenser. The solution was then heated up to 60° C. for 18 hours. Analysis by NMR showed the reaction to be complete. The solvent and residual allyl methacrylate were removed under reduced pressure at room temperature. The product was taken up in 50 ml of dry toluene and stored at −15° C. ¹H NMR spectroscopic data for D₄^{AM} is shown in Table 1.

### Example 1B

### Preparation of a Cyclotetrasiloxane Monomer Functionalized by Allyl Benzene (D₄^{AB})

9.746 g of D₄^{H} was dissolved in 10 ml of dry toluene in a round bottom flask equipped with an air condenser and a drying tube. To this solution was added 0.202 g of Karstedt's catalyst ([Pt]=3.4×10⁻⁵ mol/ml). The solution was heated while stirring to 50° C. A solution of 24.64 g allylbenzene in 45 ml of dry toluene was added at such a rate as to maintain an internal temperature of 58-60° C. After the addition, the reaction was stirred for an additional 1 h and then cooled to room temperature. 2.0 g of activated carbon was added and the mixture was allowed to stir for 45 minutes. The suspension was filtered through Celite and the solvent was removed under reduced pressure to obtain the crude product that was then re-dissolved in 10 ml of dry toluene and precipitated by pouring into 250 ml of methanol with stirring. Then the precipitate was allowed to settle and the supernatant was decanted. The precipitate was dried to constant mass to obtain the product as a colourless oil (15.911 g). ¹H NMR spectroscopic data for D₄^{AB} is shown in Table 1.

### Examples 1C to 1J

Additional functionalised cyclic monomers are shown in Table 1. Those of ordinary skill in the art would know that these products could be prepared using a variety of catalysts and in a range of different temperatures. Typically the functionalised cyclic monomers were prepared in toluene using a small excess of the allyl derivative (usually 4.5 molar equivalents to 1 mole of D₄^{H}) at room temperature to 70° C. with a suitable catalyst (usually a Pt catalyst such as PtCl₆.H₂O or Karstedt's catalyst).

Reagents for examples 1H and 1J were prepared as follows:

### Synthesis of Allyl Phenol for Use in the Synthesis of Example 1H

A solution of Boron tribromide (3.3 ml, 0.035 mol) in dichloromethane (40 ml) was added dropwise to the solution of 4-allylanisole (4.00 g, 0.0269 mol) in dichloromethane (45 ml) which has been cooled to −76° C. in an acetone/dry ice bath. The reaction mixture was allowed to warm to room temperature and stirred for 24 hours. The mixture was diluted with dichloromethane (20 ml) then cooled to −76° C. before adding saturated sodium carbonate solution and adjusted the pH to 7-8, water (30 ml) was added to aid mixing.

The mixture was extracted with dichloromethane and solids removed by filtration. The organic fraction was washed with saturated sodium chloride solution, dried over magnesium sulfate, filtered and solvent removed to give dark brown oil, 3.09 g, 83%. The crude mixture contained 2 products and no purification was attempted.

### Synthesis of Isocyanatoethylmethacrylate Derivative of Eugenol for Use in the Synthesis of Example 1J

Dibutyl tindilurate (100 μl, 23 mg/ml in toluene) was added to a solution of eugenol (5.00 g, 0.0305 mol) and isocyanatoethylmethacrylate (4.74 g, 0.0305 mol) in toluene (50 ml, dried over CaH₂). The reaction mixture was stirred at room temperature for 9 days after which it was added dropwise into 600 ml of n-pentane and the precipitate was collected under vacuum filtration to obtain a white powder, 8.65 g (89%).

TABLE 1
Examples 1A to 1J showing ¹H NMR chemical shifts of functionalised
cyclic siloxane monomers

| Ex- | | | | | | | | |
|---|---|---|---|---|---|---|---|---|
| am- | | | | | | | | |
| ple | R = | 1 | 2 | 3 | 4 | 5 | 6 | 7+ |
| 1A | 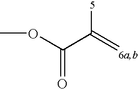 | 0.0960 (m) | 0.5637 (m) | 1.6862 (m) | 4.0701 (m) | 1.9140 (s) | a: 5.5163 (s) b: 6.0708 (s) | |
| 1B | 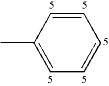 | 0.0910 (m) | 0.6024 (m) | 1.7082 (m) | 2.6530 (m) | 7.0951- 7.3724 (m) | | |
| 1C | 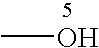 | 0.0423 (m) | 0.484 (m) | 1.5367 (m) | 3.4606 (t) | 4.2764 (s) | | |
| 1D | 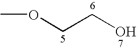 | 0.0580 (m) | 0.5106 (m) | 1.6127 (m) | 3.4061 (t) | 3.4968 (t) | 3.6788 (m) | 2.7751 (s) |
| 1E | 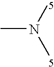 | 0.0766 (m) | 0.4594 (m) | 0.8919 (m) | 1.4209 (m) | 2.1200 (s) | | |
| 1F | 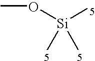 | 0.0077 (m) | 0.4290 (m) | 1.4956 (m) | 3.4520 (t) | 0.0347 (m) | | |
| 1G | 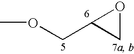 | 0.0785 (m) | 0.3724 (m) | 1.4555 (m) | 3.2706 (m) | 3.1775 (q) | 2.9388 (m) | a: 2.4026 (q); b: 2.5835 (t) |
| 1H | 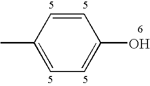 | 0.0674 | 0.5267 | 1.5739 | 2.4649 | 6.5-7.2 | 0.8239 | |
| 1I | 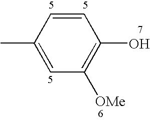 | 0.0630 (m) | 0.5655 (m) | 1.6302 (m) | 2.5411 (m) | 6.5296- 6.8968 (m) | 3.8245 (m) | 5.5371 (s) |
| 1J | 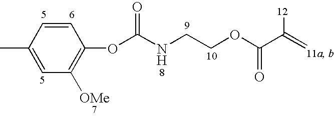 | 0.0574 (m) | 0.5619 (m) | 1.6437 (m) | 2.5756 (m) | 6.431- 6.7879 (m) | 6.9193- 7.0204 (m) | 3.7826 (m); 8 5.4034 (bs); 9 3.5635 (m); 10 4.2719 (t); 11 a: 5.5984 (s) b: 6.1412 (s); 12 1.9546 (s) |

### Example 2

### Ring Opening Polymerization (ROP) of Functional Cyclic Siloxanes

Functional cyclic siloxanes were subjected to ring opening polymerization in the presence of octamethylcyclotetrasiloxane (D₄) to obtain desired polysiloxanes with polymerizable and refractive index modifying groups. Different end groups were introduced using a variety of end group blockers.

The ROP occurs under different conditions by using a range of catalysts, which include, but are not limited to, type of base, acid, Lewis acid, and exchange resin.

The procedure is illustrated in the following scheme, in which R is Z or RIM:

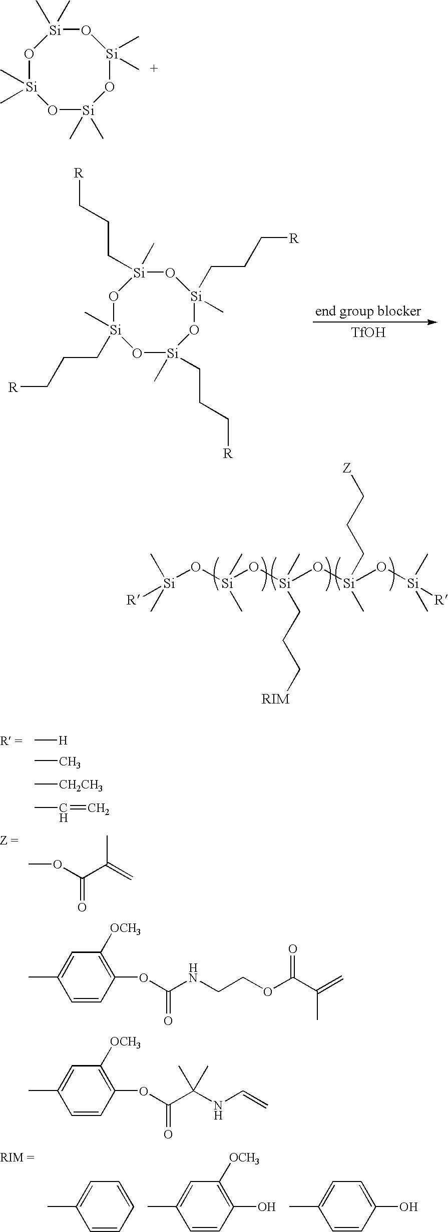

### Example 2T

### Preparation by ROP of a Copolymer of Dimethylsiloxane, Methyl Phenylpropylsiloxane, and Methyl Propylmethacrylate Siloxane, with Trimethylsilyl End Groups

A stock solution was made of 8.00 g hexamethyldisiloxane in 270.34 g D₄. 1.78 g of 2,4,6,8-tetramethyl-2,4,6,8-tetra(propyl-3-phenyl)cyclotetrasiloxane, 39.8 mg 2,4,6,8-tetramethyl-2,4,6,8-tetra(propyl-3-methyacrylatel)cyclotetrasiloxane, 2.69 g D₄, and 0.079 g of the hexamethyldisiloxane stock solution were mixed together with 1.56 g of dry toluene in a 25 ml round bottom flask under an argon atmosphere. 50 μl of trifluoro-methanesulfonic acid was quickly added whist stirring and the flask immediately covered with aluminium foil to exclude light. The reaction mixture was left stirring for 5 days. The mixture was then diluted with 5 ml toluene and neutralised with 250 mg of sodium carbonate after which the solids was filtered off and solvent removed. The crude mixture was purified by precipitation by redissolving in 5 ml toluene and added drop wise to 40 ml of ethanol whilst stirring. The precipitate was allowed to settle overnight and the supernatant decanted. The precipitation steps were repeated as necessary. All solvents were removed under reduced pressure to obtain a clear and viscous oil. It was found to have viscosity of 14550 cSt, Mn 52100, Mw 89034. The polymer contains 80.86 mol % dimethylsiloxane, 18.81 mol % methyl phenylpropylsiloxane, and 0.33 mol % methyl propylmethacrylate siloxane as determined by ¹H NMR.

### Example 2Y

### Preparation by ROP of D₄, D₄^{AB} and D₄^{EU-IEM}

A stock solution was made of 9.18 g 1,3-divinyl-1,1,3,3-tetramethyldisiloxane in 270.34 g D₄. 0.369 g of D₄^{EU-IEM} from example 1J, 3.615 g of D₄^{AB} from example 1B, and 0.35 g of the 1,3-divinyl-1,1,3,3-tetramethyldisiloxane stock solution were mixed together in a 25 ml round bottom flask under N₂ atmosphere. 200 μl of trifluoromethanesulfonic acid was quickly added whilst stirring and the flask immediately covered with aluminium foil to exclude light. The reaction mixture was heated to 70° C. for 1.5 hours then left stirring at room temperature for a further 16 hours. The mixture was diluted with 5 ml of dry toluene, added 300 mg of Na₂CO₃, stirred for 3 hours, filtered and concentrated. The residue was redissolved in 3 ml of toluene and precipitated in methanol (50 ml). The product was allowed to settle overnight, supernatant decanted and solvents removed to obtain a clear and viscous oil, 1.23 g. The composition of the copolymer was as follows: Dimethylsiloxane 77.80 mol %, methylphenylpropylsiloxane 21.45 mol % and methyleugenol-IEM siloxane 0.75 mol % with Mw of 38517, Mn 20225 and refractive index 1.4553.

### Example 2AA

### Preparation of a siloxane copolymer by ROP of D₄, D₄^{H}, and D₄^{AB}

A stock solution was prepared of 9.18 g 1,3-divinyl-1,1,3,3-tetramethyldisiloxane in 270.34 g D₄. Another stock solution was prepared of 7.24 g D₄H in 92.47 g D₄. 1.009 g of the 1,3-divinyl-1,1,3,3-tetramethyldisiloxane stock solution, 0.30 g of the D₄H stock solution and 1.74 g D₄^{AB} from example 1B were mixed in 10 ml of anhydrous toluene. 14.7 μl of trifluoro-methanesulfonic acid was added and the mixture was allowed to stir at ambient temperature for 3 days. 2.0 g anhydrous Na₂CO₃ was then added and allowed to stir at ambient temperature for 16 hours. The mixture was filtered through glass paper on a sintered glass filter. The product was precipitated by pouring the filtrate into 40 ml ethanol with vigorous stirring. The product was allowed to settle and the supernatant was decanted. The residual solvent was removed under vacuum to obtain the product as a clear and colourless oil (5.36 g).

This product is an intermediate suitable for further hydrosilylation reactions with reagents bearing polymerisable groups in order to form macromonomers of the present invention.

### Examples 2A to 2AD

A wide variety of macromonomers can be simply prepared by ring opening one or more of the functionalized cyclic monomers prepared in examples 1J to 1M. Those of ordinary skill in the art would know that these products could be prepared using a variety of catalysts and in a range of different temperatures. Typically the ring opening polymerizations are performed under acidic conditions (eg H₂SO₄, trifluoromethanesulfonic acid, trifluoromethanesulfonic acid in acetic anhydride) in toluene or as neat mixtures at room temperature to 110° C. Typically, trifluoromethanesulfonic acid is used in the range of 60-200 μl/3.5 g D₄.

The details of starting materials and the resulting macromonomers of various examples are set out in Tables 2 and 3 respectively.

Examples 2A to 2J, which illustrate macromonomers that do not contain polymerizable groups along the backbone, illustrate that polymers with high refractive index can be prepared by this methodology. Structurally similar polymers with polymerizable groups along the backbone could be prepared by the addition of suitable cyclic monomer (eg D₄^{AM}) into the polymerisation as in examples 2K to 2Y.

Examples 2Z to 2AD illustrate intermediate macromonomers suitable for further reactions with reagents bearing polymerisable groups, such as described in Schemes 8 and 10 above, in order to form macromonomers of the present invention.

TABLE 2
Mass of starting materials for examples 2A to 2AD

| Exam- | Mass of starting materials (g) | | | | | | |
|---|---|---|---|---|---|---|---|
| ple | | | | | | | mass |
| No. | End group | mass | D₄ | D₄^{AB} | D₄^{AM} | Other | (g) |
| 2A | DVTMDS | 0.0025 | 2.23 | 1.78 | | | |
| 2B | DVTMDS | 0.0023 | 2.23 | 1.34 | | | |
| 2C | DVTMDS | 0.0023 | 2.23 | 1.10 | | | |
| 2D | DVTMDS | 0.0022 | 2.23 | 0.94 | | | |
| 2E | DVTMDS | 0.0021 | 2.23 | 0.59 | | | |
| 2F | HEDS | 0.0034 | 3.14 | 2.01 | | | |
| 2G | HEDS | 0.0064 | 1.57 | 0.94 | | | |
| 2H | HEDS | 0.0269 | 1.77 | 4.23 | | | |
| 2I | HEDS | 0.0278 | 0.75 | 5.44 | | | |
| 2J | HEDS | 0.0272 | 0 | 6.00 | | | |
| 2K | DVTMDS | 0.0110 | 3.82 | 2.29 | 0.030 | | |
| 2L | DVTMDS | 0.0263 | 3.57 | 2.30 | 0.052 | | |
| 2M | DVTMDS | 0.0010 | 1.43 | 0.92 | 0.0096 | | |
| 2N | DVTMDS | 0.0013 | 1.44 | 0.92 | 0.0096 | | |
| 2O | DVTMDS | 0.0014 | 1.44 | 0.92 | 0.0096 | | |
| 2P | HMDS | 0.0009 | 1.43 | 0.92 | 0.0096 | | |
| 2Q | HMDS | 0.0009 | 1.43 | 0.92 | 0.0096 | | |
| 2R | HMDS | 0.0012 | 1.79 | 1.15 | 0.015 | | |
| 2S | HMDS | 0.0016 | 1.79 | 1.51 | 0.154 | | |
| 2T | HMDS | 0.0023 | 2.77 | 1.78 | 0.040 | | |
| 2U | HMDS | 0.0050 | 2.86 | 1.78 | 0.040 | | |
| 2V | HEDS | 0.0089 | 1.30 | 0.84 | 0.014 | | |
| 2W | HEDS | 0.1186 | 18.25 | 12.00 | 0.27 | | |
| 2X | DVTMDS | 0.0038 | 1.26 | 0.71 | | D₄^{EU-IEM} | 0.02 |
| 2Y | DVTMDS | 0.0115 | 3.95 | 2.60 | | D₄^{EU-IEM} | 0.37 |
| 2Z | HMDS | 0.0011 | 1.19 | 0.71 | | D₄^{EU} | 0.23 |
| 2AA | DVTMDS | 0.0330 | 8.20 | 1.74 | | D₄^{H} | 0.02 |
| 2AB | DVTMDS | 0.0330 | 11.74 | 7.79 | | D_{H}^{AA} | 0.43 |
| 2AC | DVTMDS | 0.0330 | 12.15 | 7.79 | | D₄^{H} | 0.02 |
| 2AD | DVTMDS | 0.3750 | 11.18 | 7.00 | | D₄^{EU} | 0.11 |

TABLE 3
Molar percentages and characteristics of macromonomers of examples
2A to 2AD

| Example | | Mol % in product by ¹H nmr | | | | | GPC | | | RI (@ | Viscosity |
|---|---|---|---|---|---|---|---|---|---|---|---|
| No. | End group | D₄ | D₄^{AB} | D₄^{AM} | Other | Mol % | Mw | Mn | PD | 37° C.) | (cSt) |
| 2A | DVTMDS | 79.5 | 20.5 | | | | 17380 | 11516 | 1.51 | 1.44969 | |
| 2B | DVTMDS | 83.6 | 16.4 | | | | 16958 | 9360 | 1.81 | 1.44107 | |
| 2C | DVTMDS | 86.8 | 13.2 | | | | 19580 | 11743 | 1.67 | 1.43508 | |
| 2D | DVTMDS | 88.2 | 11.8 | | | | 22490 | 12050 | 1.87 | 1.43212 | |
| 2E | DVTMDS | 92.8 | 7.2 | | | | 18138 | 13060 | 1.39 | 1.41964 | |
| 2F | HEDS | 81.0 | 19.0 | | | | 90628 | 40022 | 2.26 | | |
| 2G | HEDS | 82.3 | 17.7 | | | | 36237 | 25892 | 1.40 | 1.4444 | |
| 2H | HEDS | 50.8 | 49.2 | | | | 12657 | 6667 | 1.90 | 1.4876 | |
| 2I | HEDS | 27.7 | 72.3 | | | | 5058 | 3209 | 1.58 | 1.50711 | |
| 2J | HEDS | 4.3 | 95.7 | | | | 3334 | 2408 | 1.38 | 1.52271 | |
| 2K | DVTMDS | 89.26 | 10.47 | 0.27 | | | 68462 | 27395 | 2.50 | 1.4310 | |
| 2L | DVTMDS | 82.52 | 16.28 | 1.20 | | | 65933 | 31180 | 2.11 | 1.44333 | 1840 |
| 2M | DVTMDS | 81.69 | 17.92 | 0.39 | | | 45773 | 21879 | 2.09 | 1.44596 | 1560 |
| 2N | DVTMDS | 82.05 | 17.75 | 0.20 | | | 33355 | 18061 | 1.85 | 1.44564 | 930 |
| 2O | DVTMDS | 82.07 | 17.75 | 0.18 | | | 38240 | 21197 | 1.80 | 1.44556 | 1140 |
| 2P | HMDS | 81.58 | 18.15 | 0.27 | | | 26239 | 16262 | 1.61 | 1.4466 | 320 |
| 2Q | HMDS | 79.9 | 19.9 | 0.20 | | | 28271 | 17532 | 1.61 | 1.44584 | |
| 2R | HMDS | 80.15 | 19.55 | 0.30 | | | 95813 | 48231 | 1.99 | 1.44528 | |
| 2S | HMDS | 80.08 | 19.80 | 0.12 | | | 61494 | 39001 | 1.58 | 1.44775 | 1910 |
| 2T | HMDS | 80.86 | 18.81 | 0.33 | | | 89034 | 52100 | 1.71 | | 14550 |
| 2U | HMDS | 79.6 | 20.2 | 0.25 | | | 89839 | 51575 | 1.74 | | 13810 |
| 2V | HEDS | 80.2 | 19.6 | 0.20 | | | 25193 | 16864 | 1.49 | | |
| 2W | HEDS | 78.7 | 21.0 | 0.34 | | | 95597 | 53927 | 1.77 | 1.44797 | |
| 2X | DVTMDS | 83.91 | 15.96 | | D₄^{EU-IEM} | 0.13 | 32450 | 17943 | 1.8 | | |
| 2Y | DVTMDS | 77.80 | 21.45 | | D₄^{EU-IEM} | 0.75 | 38517 | 20225 | 1.9 | 1.4553 | |
| 2Z | HMDS | 77.84 | 18.73 | | D₄^{EU} | 3.43 | | | | 1.45783† | |
| 2AA | DVTMDS | 95.28 | 4.47 | | D₄^{H} | 0.25 | 54 095 | 35 621 | 1.52 | 1.41856‡ | 4 470 |
| 2AB | DVTMDS | 52.5 | 45.7 | | D₄^{AA} | 1.8 | 20 527 | 12 290 | 1.67 | | |
| 2AC | DVTMDS | 39.3 | 59.1 | | D₄^{EU} | 1.6 | 11 425 | 5 327 | 2.14 | | |
| 2AD | DVTMDS | 79.7 | 20.10 | | D₄^{H} | 0.25 | 44 889 | 35 283 | 1.27 | | |

†21.3° C.;
‡19.4° C.

### Example 3

### Synthesis of Silane Functionalised Prepolymers

In examples 3A to 3D silane functionalized prepolymers were prepared by ring opening polymerization of D₄ with D₄^{H} as shown in the following scheme. The ratio of silane functional groups along the backbone was controlled to afford modification with polymerizable and refractive index modifying groups in later steps. Different end groups are introduced by using a variety of end group blockers. The ROP occurs under different conditions by using a range of catalysts, which include, but are not limited to, type of acid, Lewis acid, and exchange resin.

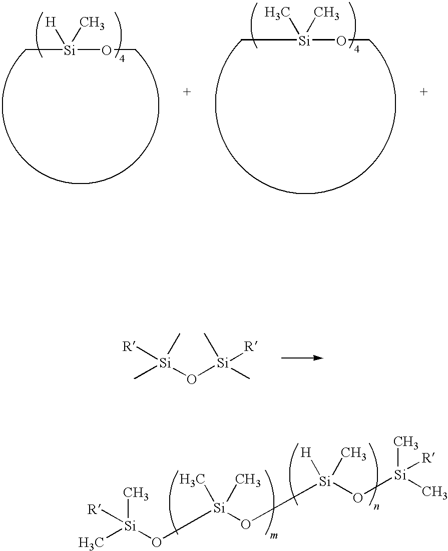

### Example 3B

### Preparation of Siloxane Copolymer Containing 20-30 mol % Silane Functional Groups

1.003 g HMDS, 44.205 g D₄^{H} and 129.03 g D₄ were dissolved in 200 ml toluene. 260 μl trifluoro-methanesulfonic acid was added. The solution was allowed to stir at ambient temperature for 7 days. 25.0 g anhydrous sodium carbonate was added and the mixture was allowed to stir at ambient temperature for 3 hours. The mixture was then filtered through glass filter paper on a sintered glass filter. The filtrate was added drop-wise to 400 ml ethanol. The supernatant was decanted and the residue was evaporated under vacuum to obtain a clear colourless oil (104.108 g).

TABLE 4
Summary of results of examples 3A to 3D

| Example | | SiH mol % | ¹H NMR data | | |
|---|---|---|---|---|---|
| number | R′ = | by ¹H NMR | Si(CH₃)₂ | SiH | Viscosity (cSt) |
| 3A | CH₃ | 19.6 | 0.069 | 4.68 | 16100 |
| 3B | CH₃ | 28.0 | 0.069 | 4.68 | 880 |
| 3C | CH₃ | 28.18 | 0.069 | 4.68 | 2610 |
| 3D | CH₃ | 29.0 | 0.069 | 4.68 | 853 |

### Example 4

### Functionalization of Silane Prepolymers

The prepolymers prepared in examples 3A to 3D were functionalized by allyl compounds via hydrosilylation to introduce polymerizable groups and refractive index modifying groups in one or two steps. The hydrosilylation is illustrated in the following scheme.

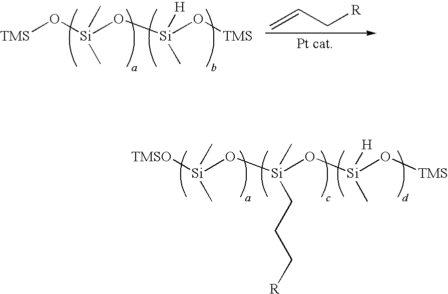

### Example 4E

### Functionalization of a Silane Prepolymer with Allylbenzene

3.007 g of 28 mol % silane copolymer (example 3B) was dissolved in 20 ml of toluene in a 50 ml round bottom flask equipped with a condenser. 1.034 g of allylbenzene (AB) was added, followed by 100 μl of Karstedt's catalyst solution in toluene ([Pt]=3.4×10⁻⁵ M). The solution was stirred at 40° C. under N₂ for 18 hours. An aliquot was removed and dried to give a clear and viscous oil. ¹H NMR analysis showed that the resultant polymer contains 11.38 mol % Si—H, 17.32 mol % allylbenzene and 71.30 mol % dimethyl groups. This allybenzene functionalised copolymer was not isolated, instead it was used as an intermediate for the preparation of example 4J.

Additional allylbenzene functionalized silane prepolymers were prepared in examples 4A to 4H, the results of which are set out in Table 5.

### Example 4J

### Functionalization of a Silane Prepolymer with Allyl Alcohol

4.041 g of the silane prepolymer of Example 4E was dissolved in 20 ml of toluene. 2.241 g of allyl alcohol (AA) was added followed by 100 μl of Karstedt's catalyst solution in toluene ([Pt]=3.4×10⁻⁵ M). The solution was heated at 40° C. for 19 hours. The solution was cooled to room temperature and 1.50 g of activated carbon was added. The mixture was stirred for 3 hours, then filtered through glass filter paper on a glass sintered filter, followed by filtration through a 0.22 μm hydrophobic PVF filter. The product was found to contain 0.55 mol % Si—H, 10.72 mol % allylalcohol; 17.01 mol % allylbenzene and 72.65 mol % dimethyl.

The allyl alcohol functionalized silane prepolymer may be reacted with reagents containing polymerisable groups to form macromonomers of the present invention.

Additional functionalized silane prepolymers were prepared in examples 4I and 4K, the results of which are set out in Table 5.

TABLE 5
Summary of results of examples 4A to 4K

| | | Prepolymer | | | mol % by ¹H | |
|---|---|---|---|---|---|---|
| Example | R = | (g) | R (g) | Catalyst (μl) | NMR | RI(23° C.) |
| 4A | 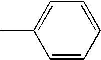 | 5.011 | 7.494 | H2PtCl6, 160 | 6.00% AB; 22% Si—H | |
| 4B | 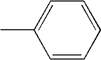 | 3.032 | 4.613 | H2PtCl6, 100 | 8.5% AB; 20% Si—H | |
| 4C | 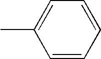 | 3.066 | 1.271 | Karstedt's, 100 | 9.5% AB; 19% Si—H | 1.43168 |
| 4D | 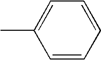 | 3.021 | 0.089 | Karstedt's, 120 | 12.98% AB; 14.91% Si—H | 1.44428 |
| 4E | 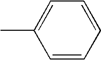 | 3.007 | 1.034 | Karstedt's, 100 | 17.32% AB; 11.38% Si—H | |
| 4F | 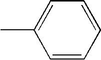 | 3.036 | 1.030 | Karstedt's, 120 | 18.73% AB; 10.98% Si—H | 1.4484 |
| 4G | 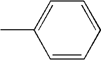 | 3.001 | 1.148 | Karstedt's, 120 | 20.87% AB; 8.90% Si—H | 1.45272 |
| 4H | 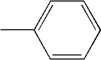 | 3.053 | 1.275 | Karstedt's, 120 | 21.15% AB; 6.3% Si—H | 1.45484 |
| 41 | —OH | 2.012 | 0.158 | Karstedt's, 100 | 5.88% AA; 20.9% AB | |
| 4J | —OH | 4.041 | 2.241 | Karstedt's, 100 | 10.72% AA; 17.01% AB | |
| 4K | 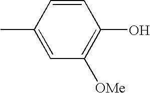 | 3.007 | 6.325 | Karstedt's, 100 | 29.20% Eu | 1.48548 |

### Example 5

### Preparation of Siloxane Methacrylate from Siloxane Eugenol Derivative and IEM

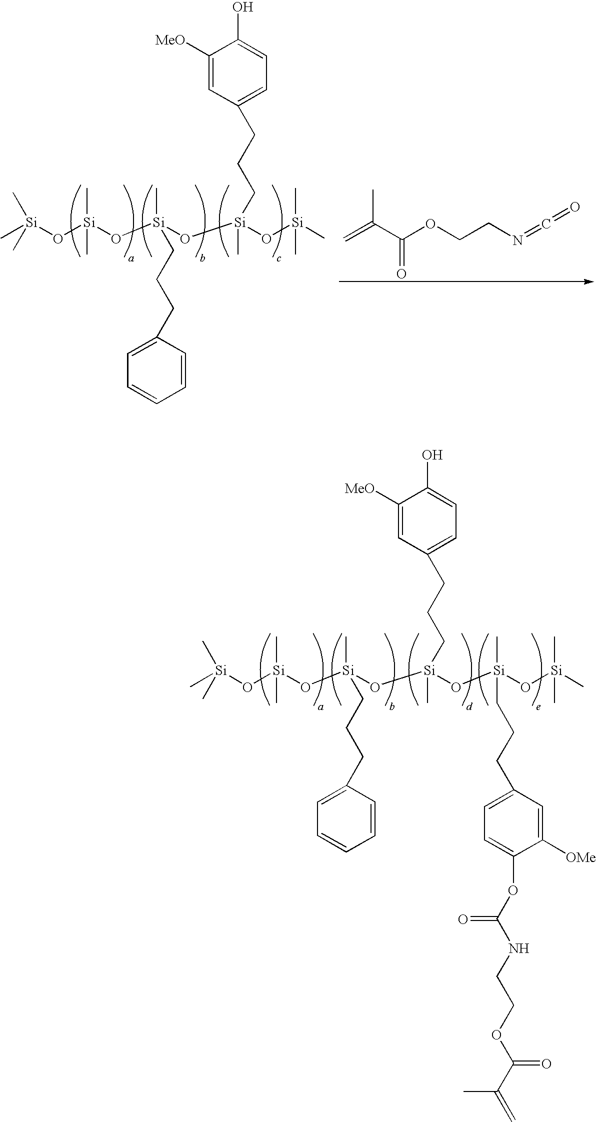

Isocyanatoethylmethacrylate (4.66 g of a 0.230 g IEM in 21.69 g of toluene), allyl benzene and eugenol functionalized polymer (0.880 g; a=77.8%, b=18.7%, c=3.5%; RI=1.4578 at 21° C.), and dibutyltindilaurate (25 μl) were mixed and stirred at room temperature for 17 h. The reaction mixture was precipitated into methanol. The precipitated polymer was collected and evaporated to dryness to afford an oil (0.883 g). ¹H NMR analysis gave the desired IEM functionalized macromonomer with the following molar percentage ratio: a=79.3, b=17.0, d=1.0, e=2.7. Refractive index of the polymer was 1.458 at 21° C.

### Example 6

### Functionalization of Silane Prepolymers by Polymerizable and Refractive Index Modifying Groups Via a Mixed Hydrosilylation

A mixed hydrosilylation in one pot synthesis is shown in the following scheme:

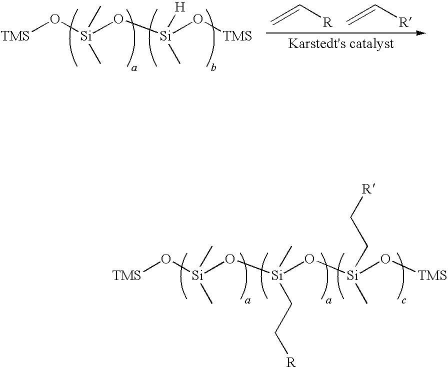

### Example 6C

### Functionalization of a Silane Prepolymer with Allyl Benzene and Eugenol (13:1)

3.01 g of silane prepolymer containing 28 mol % silane groups (example 3B), 5.69 g of allylbenzene and 0.637 g of eugenol were dissolved in 25 ml toluene in a 50 ml round bottom flask equipped with a condenser and gas inlet tap under N₂. 100 μl of Karstedt's catalyst solution in toluene ([Pt]=3.4×10⁻⁵ M) was added to the solution and the mixture was stirred at 40° C. under N₂ and monitored by ¹H NMR until all the Si—H groups were consumed. The mixture was then cooled to room temperature, followed by addition of 0.300 g of activated carbon and stirred for 3 hours after which the carbon was filtered off. The solvent was removed from the filtrate and the product was taken up in 10 ml of n-pentane and washed with saturated NaHCO₃ (2×30 ml); water (30 ml) then saturated NaCl (30 ml) and dried over MgSO₄. The product was dried under reduced pressure to yield a clear, slightly yellow and viscous oil, 3.492 g. The polymer was found to contain 26.05 mol % allylbenzene; 2.0 mol % eugenol and 71.95 mol % dimethylsiloxane groups as determined by ¹H NMR and the refractive index is 1.47272 (23.4° C.).

The silane prepolymer may be reacted with reagents containing polymerisable groups to form macromonomers of the present invention.

Additional examples 6A to 6F are shown in Table 6. Again, the prepolymers in examples 6A, 6B, 6C, 6E and 6F may be reacted with reagents containing polymerisable groups to form macromonomers of the present invention.

TABLE 6
Details and results of Examples 6A to 6F

| | | | | | Mass | | |
|---|---|---|---|---|---|---|---|
| | Mass | | Mass first | | second | | |
| Ex- | pre- | | allyl | | allyl | | Functionalisa- |
| am- | polymer | | derivative | | derivative | | tion mol % |
| ple | (g) | R = | (g) | R′ = | (g) | RI | by ¹H NMR |
| 6A | 2.00 | 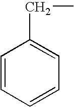 | 3.79 | 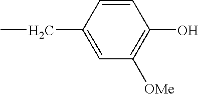 | 0.21 | | 14.6 mol % AB and 6.3 mol % EU |
| 6B | 2.00 | 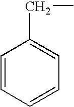 | 3.79 | 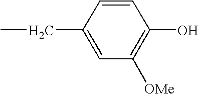 | 0.10 | | 10.4 mol % AB and 1.2 mol % EU |
| 6C | 3.01 | 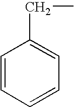 | 5.69 | 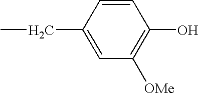 | 0.637 | 1.4727 | 26.05 mol % AB and 2.0 mol % EU |
| 6D | 2.00 | 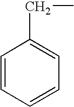 | 3.79 | 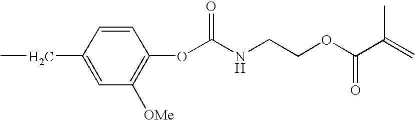 | 0.10 | | 10.67 mol % AB and 0.23 mol % EU |
| 6E | 2.00 | 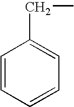 | 3.79 | —CH₂OH | 0.037 | 1.4465 | 9.7 mol % AB and 2.2 mol % AA |
| 6F | 2.00 | 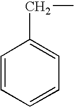 | 3.79 | —CH₂OH | 0.019 | 1.4460 | 12.2 mol % AB and 3.8 mol % AA |

It will be understood that the invention disclosed and defined in this specification extends to all alternative combinations of two or more of the individual features mentioned or evident from the text or drawings. All of these different combinations constitute various alternative aspects of the invention.

1. A macromonomer of the formula I: 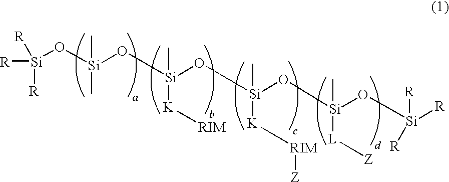
wherein
RIM is a refractive index modifying group;
Z is a free radically polymerisable group;
K is a spacer group;
L is optional and is a spacer group;
each R is independently selected from an RIM, a lower alkyl group, hydrogen or Z;
a is a molar percentage of the macromonomer which is in the range of from 0 to 95 mol %;
b is a molar percentage of the macromonomer which is in the range of from 5 to 99 mol %;
c is a molar percentage of the macromonomer which is in the range of from 0 to 2 mol %; and
d is a molar percentage of the macromonomer which is in the range of from 0 to 2 mol %;
with the proviso that c and d are not both 0 mol %.

2. A macromonomer according to claim 1 wherein each RIM is independently selected from the group consisting of a substituted or unsubstituted aromatic group, a fluorinated group, a group containing bromine, iodine, or chlorine atom(s) and a sulphur containing group.

3. A macromonomer according to claim 2 wherein each RIM is a substituted or unsubstituted phenyl ring.

4. A macromonomer according to claim 1 wherein each Z is an ethylenically unsaturated group.

5. A macromonomer according to claim 1 wherein each K is independently selected from the group consisting of a linear, branched, or cyclic lower alkyl, which is optionally interrupted by one or more heteroatoms or substituted by one or more of an ester, amide, urethane, carbonate, thioester or —C(S)—NH—.

6. A macromonomer according to claim 5 wherein each K is a lower alkyl of the formula —(CH₂)n-, wherein n is an integer 1, 2, 3, 4 or 5.

7. A macromonomer according to claim 1 wherein each L is a lower alkyl of the formula —(CH₂)n-, wherein n is an integer 1, 2, 3, 4 or 5.

8. A macromonomer according to claim 1 having a refractive index at 37° C. is in the range of from greater than 1.33 to 1.60.

9. A macromonomer according to claim 1 having a viscosity at 25° C. of less than 150,000 cSt.

10. A macromonomer according to claim 1 having, when cured into a polymer, a modulus at 37° C. of less than 50 kPa.

11. A composition curable into a biomedical device including a macromonomer according to claim 1.

12. (canceled)

13. An in situ curable, accommodating intraocular lens formed from the composition of claim 11.

14. A method of producing in situ an intraocular lens including the steps of introducing a composition according to claim 11 into a lens capsular bag and curing the composition.

15. (canceled)
